(19) United States
(12) Patent Application Publication
(10) Pub. No.: US 2023/0416765 A1
(43) Pub. Date: Dec. 28, 2023

(54) AGROBACTERIUM RHIZOGENES AND METHODS OF TRANSFORMING CELLS

(71) Applicant: Pairwise Plants Services, Inc, Durham, NC (US)

(72) Inventors: Fang-Ming Lai, Cary, NC (US); Eric Dean, Raleigh, NC (US); Ming Cheng, Cary, NC (US); Joel Reiner, Raleigh, NC (US); Qingchun Shi, Cary, NC (US); David Schwark, New Hill, NC (US); Sharon Leigh Guffy, Durham, NC (US); Aaron Hummel, Hillsborough, NC (US)

(21) Appl. No.: 18/253,494

(22) PCT Filed: Nov. 23, 2021
(86) PCT No.: PCT/US2021/060480

§ 371 Date: May 18, 2023

Related U.S. Application Data
(60) Provisional application No. 63/117,173, filed on Nov. 23, 2020.

(51) Int. Cl. C12N 15/82

(52) U.S. Cl. CPC C12N 15/8205

(57) ABSTRACT

Agrobacterium rhizogenes are described herein along with methods of transforming cells and methods of using Agrobacterium rhizogenes and/or transformed cells. Also described herein are plants, such as plants in the Rubus family, including a modified nucleic acid, optionally wherein the plants are transgene-free and include a modified nucleic acid.

### STATEMENT REGARDING ELECTRONIC FILING OF A SEQUENCE LISTING

A Sequence Listing in ASCII text format, submitted under 37 C.F.R. § 1.821, entitled 1499-54WO_ST25, 1,184,886 bytes in size, generated on Nov. 21, 2021, and filed via EFS-Web, is provided in lieu of a paper copy. This Sequence Listing is hereby incorporated herein by reference into the specification for its disclosures.

### FIELD

This invention relates to Agrobacterium rhizogenes, to methods of transforming cells, and to methods of use of Agrobacterium rhizogenes and/or transformed cells. The invention further relates to plants, such as plants in the Rubus family, including a modified nucleic acid, optionally transgene-free plants including a modified nucleic acid.

### BACKGROUND

Successful genetic transformation in plant species depends on delivering T-DNA into regeneration-competent cells and the subsequent regeneration of a plantlet from those transgenic cells. Various Agrobacterium rhizogenes strains have been used for producing transgenic roots and full transgenic plants via transgenic roots (Tepfer, D., Physiol Plant 79: 140-146, 1990). For example, successful regeneration of full transgenic plants from transgenic hairy roots was reported in Centarurium erythraea (Subotic et al. Biol Plant 47(4): 617-619, 2003). Infection by wild-type Agrobacterium rhizogenes and subsequent formation of abnormal roots (hairy roots) was reported in Vitis and Rubus (Hemstad, P. R. and Reisch, B. I., J. Plant Physiol. 120(1): 9-17 (1985). However, there are no reports or disclosures on generating transgenic roots carrying T-DNA from the tumor inducing (Ti) plasmid and full transgenic plants from transgenic roots in Rubus.

There are reports on generating composite plants which contain transgenic roots and wild-type aerial parts using Agrobacterium rhizogenes, for example, in peanut (Geng et al., Plant Cell Tiss Organ Cult 109: 491-500, 2012) and peach (Xu et al., Scientific Reports 10: 2836, 2020). The composite plants in these reports were used for genetic studies of roots and gene functionality, but offspring were not able to inherit the transgene.

Agrobacterium rhizogenes strains applied to plant genetic transformation contain the hairy root inducing genes (e.g., rol genes) and may lead to hairy root phenotypes such as short internodes, wrinkled leaves, abundant roots with extensive lateral branching, and increased production of certain metabolites (Tepfer Cell 47: 957-967 (1984) and Shen et al. Proc. Natl. Acad. Sci. USA 85, 3417-3421; 1988). A. rhizogenes strains can be disarmed by removing the T-DNA from its Ri plasmid while maintaining the trans factors including the vir genes (Vilaine, F. and Casse-Debart, F. Mol Gen Genet 206: 17-23, 1987 and Mankin, S., et al., In Vitro Cell Dev Biol 43: 521-535, 2007). A disarmed K599 strain of A. rhizogenes can deliver the T-DNA of its Ti plasmid efficiently and produces phenotypically normal transgenic soybean plants (Mankin et al. 2007). However, there are no reports on using a disarmed Agrobacterium rhizogenes strain in Rubus transformation.

To date, an efficient and genotype-independent genetic transformation system has not yet been developed for Rubus. Although successful regeneration of transformants has been reported, these reported processes are highly genotype dependent and are mainly limited by a high level of recalcitrance for regeneration that exists within the genus. Existing reports have generally followed conventional transformation and regeneration approaches in vitro, namely, shoot induction from the explants, shoot development and then the root induction, and these transformation processes require a lengthy duration in obtaining transgenic plantlets in vitro. For example, generation of transgenic Rubus plants has historically proven to be a difficult and time-consuming process. A duration of six months to eight months is commonly needed to generate transgenic plants in the Rubus plants. Accordingly, new transformation protocols are needed.

### SUMMARY OF EXAMPLE EMBODIMENTS

One aspect of the present invention is directed to a method of transforming a plant cell, the method comprising: introducing a polynucleotide from an Agrobacterium rhizogenes strain into the plant cell to provide a transformed plant cell, thereby transforming the plant cell. In some embodiments, the transformed plant cell is a transgenic plant cell.

Another aspect of the present invention is directed to a method of producing an edited plant, the method comprising: introducing a polynucleotide from an Agrobacterium rhizogenes strain into a plant cell of an explant to provide an edited cell, wherein the polynucleotide encodes or comprises at least a portion of an editing system that is capable of modifying a target nucleic acid in the plant cell to provide a modified nucleic acid and thereby the edited cell; producing a root from the edited cell; and producing an edited plant from the root.

A further aspect of the present invention is directed to a method of transforming a plant cell, the method comprising: introducing a polynucleotide from a disarmed Agrobacterium rhizogenes strain MSU440 into the plant cell to provide a transformed plant cell, thereby transforming the plant cell. In some embodiments, the transformed plant cell is a transgenic plant cell.

Another aspect of the present invention is directed to a method for evaluating transgene function in a plant cell, the method comprising: introducing a polynucleotide from an Agrobacterium rhizogenes strain into the plant cell to provide a transformed plant cell comprising the transgene; and evaluating the transformed plant cell to determine transgene function. In some embodiments, the transformed plant cell is a transgenic plant cell.

An additional aspect of the present invention is directed to a disarmed Agrobacterium rhizogenes strain as described herein and/or a composition comprising a disarmed Agrobacterium rhizogenes strain as described herein.

The present invention further provides expression cassettes and/or vectors comprising a nucleic acid construct of the present invention, and provides cells comprising a polypeptide and/or nucleic acid construct of the present invention. Additionally, the present invention provides kits comprising a nucleic acid construct and/or polypeptide of the present invention and expression cassettes, vectors and/or cells comprising the same.

It is noted that aspects of the present invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 is a schematic illustration of an Agrobacterium rhizogenes MSU440 T-DNA knockout cassette according to embodiments of the present invention. “Prom” is the antibiotic resistance gene promoter that is followed by an antibiotic resistance gene (e.g., tetracycline or gentamicin) (“Antibiotic Resistance CDS”), and then a terminator (“Term”). “FRT” depicts FRT recombination sites for post-knockout cassette removal by yeast FLP recombinase. “Homologous Sequence” depicts a sequence homologous to the 5′ and 3′ ends of the T-DNAs in Ri plasmid pRIA4, and “RS” depicts the unique restriction sites for rapid cloning of homologous sequences.

FIG. 2 is schematic illustration showing a suicide vector and SacB counter-selection strategy according to embodiments of the present invention.

FIG. 3 depicts the Ri plasmid of Agrobacterium rhizogenes MSU440, which includes two T-DNAs that are labeled TL-DNA and TR-DNA. Arrows point to a sequence upstream of TL-DNA and a sequence downstream of TR-DNA, which were used as homologous recombination arm sequences in the knockout cassette.

FIG. 4 depicts the Ri plasmid of Agrobacterium rhizogenes strain DS101.1.

FIG. 5 shows the alignment of the Ri plasmid from MSU440 and the Ri plasmid from DS101.1. The alignment shows that in DS101.1, both T-DNAs and the intervening sequence are absent.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

A “heterologous” or a “recombinant” nucleotide sequence is a nucleotide sequence not naturally associated with a host cell into which it is introduced, including non-naturally occurring multiple copies of a naturally occurring nucleotide sequence.

A “native” or “wild-type” nucleic acid, nucleotide sequence, polypeptide or amino acid sequence refers to a naturally occurring or endogenous nucleic acid, nucleotide sequence, polypeptide or amino acid sequence. Thus, for example, a “wild-type mRNA” is an mRNA that is naturally occurring in or endogenous to the reference organism. A “homologous” nucleic acid sequence is a nucleotide sequence naturally associated with a host cell into which it is introduced.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “recombinant nucleic acid,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement” as used herein can mean 100% complementarity with the comparator nucleotide sequence or it can mean less than 100% complementarity (e.g., “substantially complementary,” such as about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity).

A “portion” or “fragment” of a nucleotide sequence or polypeptide sequence will be understood to mean a nucleotide or polypeptide sequence of reduced length (e.g., reduced by 1, 2, 3, 4, 5, 6, 7, 8, 9, 10, 11, 12, 13, 14, 15, 16, 17, 18, 19, 20 or more residue(s) (e.g., nucleotide(s) or peptide(s)) relative to a reference nucleotide or polypeptide sequence, respectively, and comprising, consisting essentially of and/or consisting of a nucleotide or polypeptide sequence of contiguous residues, respectively, identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleotide or polypeptide sequence. Such a nucleic acid fragment or portion according to the invention may be, where appropriate, included in a larger polynucleotide of which it is a constituent. As an example, a repeat sequence of guide nucleic acid of this invention may comprise a portion of a wild-type CRISPR-Cas repeat sequence (e.g., a wild-type Type V CRISPR Cas repeat, e.g., a repeat from the CRISPR Cas system that includes, but is not limited to, Cas12a (Cpf1), Cas12b, Cas12c (C2c3), Cas12d (CasY), Cas12e (CasX), Cas12g, Cas12h, Cas12i, C2c1, C2c4, C2c5, C2c8, C2c9, C2c10, Cas14a, Cas14b, and/or Cas14c, and the like).

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis ofSequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 9100, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4×-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention can be codon optimized for expression. In some embodiments, a polynucleotide, nucleic acid construct, expression cassette, and/or vector of the present invention (e.g., that comprises/encodes a nucleic acid binding polypeptide (e.g., a DNA binding domain such as a nucleic acid binding polypeptide from a polynucleotide-guided endonuclease, a zinc finger nuclease, a transcription activator-like effector nuclease (TALEN), an Argonaute protein, and/or a CRISPR-Cas effector protein), a guide nucleic acid, a cytosine deaminase and/or adenine deaminase) may be codon optimized for expression in an organism (e.g., an animal, a plant, a fungus, an archaeon, or a bacterium). In some embodiments, the codon optimized nucleic acid constructs, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acid constructs, polynucleotides, expression cassettes, and/or vectors that have not been codon optimized.

In any of the embodiments described herein, a polynucleotide or nucleic acid construct of the invention may be operatively associated with a variety of promoters and/or other regulatory elements for expression in an organism or cell thereof (e.g., a plant and/or a cell of a plant). Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron).

By “operably linked” or “operably associated” as used herein in reference to polynucleotides, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” or “fused” in reference to polypeptides, refers to the attachment of one polypeptide to another. A polypeptide may be linked or fused to another polypeptide (at the N-terminus or the C-terminus) directly (e.g., via a peptide bond) or through a linker (e.g., a peptide linker).

The term “linker” in reference to polypeptides is art-recognized and refers to a chemical group, or a molecule linking two molecules or moieties, e.g., two domains of a fusion protein, such as, for example, a CRISPR-Cas effector protein and a peptide tag and/or a polypeptide of interest. A linker may be comprised of a single linking molecule (e.g., a single amino acid) or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

In some embodiments, a peptide linker useful with this invention may be about 2 to about 100 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140 150 or more amino acids in length). In some embodiments, a peptide linker may be a GS linker.

As used herein, the term “linked,” or “fused” in reference to polynucleotides, refers to the attachment of one polynucleotide to another. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g., extension of the hairpin structure in guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227). In some embodiments, a promoter region may comprise at least one intron (e.g., SEQ ID NO:1 or SEQ ID NO:2).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)).

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the European patent publication EP0342926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as 0-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, incorporated by reference herein for its disclosure of promoters. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); European patent EP 0452269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9(5):297-306 (2015)), ZmSTK2_USP from maize (Wang et al. Genome 60(6):485-495 (2017)), LAT52 and LAT59 from tomato (Twell et al. Development 109(3):705-713 (1990)), Zm13 (U.S. Pat. No. 10,421,972), PLA₂-δ promoter from arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHEs) (KIM ET AL. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EAMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986)Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron.

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

An “editing system” as used herein refers to any site-specific (e.g., sequence-specific) nucleic acid editing system now known or later developed, which system can introduce a modification (e.g., a mutation) in a nucleic acid in a target specific manner. For example, an editing system can include, but is not limited to, a CRISPR-Cas editing system, a meganuclease editing system, a zinc finger nuclease (ZFN) editing system, a transcription activator-like effector nuclease (TALEN) editing system, a base editing system, and/or a prime editing system, each of which may comprise one or more polypeptide(s) and/or one or more polynucleotide(s) that when present and/or expressed together in a composition and/or cell can modify (e.g., mutate) a target nucleic acid in a sequence specific manner. In some embodiments, an editing system (e.g., a site- and/or sequence-specific editing system) comprises one or more polynucleotide(s) encoding for and/or one or more polypeptide(s) including, but not limited to, a nucleic acid binding polypeptide (e.g., a DNA binding domain) and/or a nuclease. In some embodiments, an editing system is encoded by one or more polynucleotide(s).

In some embodiments, an editing system comprises one or more sequence-specific nucleic acid binding polypeptide(s) (e.g., a DNA binding domain) that can be from, for example, a polynucleotide-guided endonuclease, a CRISPR-Cas effector protein (e.g., a CRISPR-Cas endonuclease), a zinc finger nuclease, a transcription activator-like effector nuclease (TALEN) and/or an Argonaute protein. In some embodiments, an editing system comprises one or more cleavage polypeptide(s) (e.g., a nuclease) such as, but not limited to, an endonuclease (e.g., Fok1), a polynucleotide-guided endonuclease, a CRISPR-Cas endonuclease, a zinc finger nuclease, and/or a transcription activator-like effector nuclease (TALEN).

A “nucleic acid binding polypeptide” as used herein refers to a polypeptide that binds and/or is capable of binding a nucleic acid in a site- and/or sequence specific manner. In some embodiments, a nucleic acid binding polypeptide comprises a DNA binding domain. In some embodiments, a nucleic acid binding polypeptide may be a sequence-specific nucleic acid binding polypeptide such as, but not limited to, a sequence-specific binding polypeptide and/or domain from, for example, a polynucleotide-guided endonuclease, a CRISPR-Cas effector protein (e.g., a CRISPR-Cas endonuclease), a zinc finger nuclease, a transcription activator-like effector nuclease (TALEN) and/or an Argonaute protein. In some embodiments, a nucleic acid binding polypeptide comprises a cleavage polypeptide (e.g., a nuclease polypeptide and/or domain) such as, but not limited to, an endonuclease (e.g., Fok1), a polynucleotide-guided endonuclease, a CRISPR-Cas endonuclease, a zinc finger nuclease, and/or a transcription activator-like effector nuclease (TALEN). In some embodiments, the nucleic acid binding polypeptide associates with and/or is capable of associating with (e.g., forms a complex with) one or more nucleic acid molecule(s) (e.g., forms a complex with a guide nucleic acid as described herein) that can direct or guide the nucleic acid binding polypeptide to a specific target nucleotide sequence (e.g., a gene locus of a genome) that is complementary to the one or more nucleic acid molecule(s) (or a portion or region thereof), thereby causing the nucleic acid binding polypeptide to bind to the nucleotide sequence at the specific target site. In some embodiments, the nucleic acid binding polypeptide is a CRISPR-Cas effector protein as described herein. In some embodiments, reference is made to specifically to a CRISPR-Cas effector protein for simplicity, but a nucleic acid binding polypeptide as described herein may be used.

In some embodiments, an editing system comprises a ribonucleoprotein such as an assembled ribonucleoprotein complex (e.g., a ribonucleoprotein that comprises a CRISPR-Cas effector protein and a guide nucleic acid in the form of complex). A complex of an editing system may be a covalently and/or non-covalently bound complex. An editing system, as used herein, may be assembled when introduced into a plant cell (e.g., assembled into a complex prior to introduction into the plant cell) and/or may assemble into a complex (e.g., a covalently and/or non-covalently bound complex) after and/or during introduction into a plant cell. Exemplary ribonucleoproteins and methods of use thereof include, but are not limited to, those described in Malnoy et al., (2016) Front. Plant Sci. 7:1904; Subburaj et al., (2016) Plant Cell Rep. 35:1535; Woo et al., (2015) Nat. Biotechnol. 33:1162; Liang et al., (2017) Nat. Commun. 8:14261; Svitashev et al., Nat. Commun. 7, 13274 (2016); Zhang et al., (2016) Nat. Commun. 7:12617; Kim et al., (2017) Nat. Commun. 8:14406.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a nucleic acid construct of the invention (e.g., a polynucleotide encoding a CRISPR-Cas effector protein, a polynucleotide encoding a deaminase (e.g., a cytosine deaminase and/or an adenine deaminase), and/or a polynucleotide comprising a guide nucleic acid), wherein the nucleic acid construct is operably associated with at least a control sequence (e.g., a promoter). Thus, some embodiments of the invention provide expression cassettes designed to express, for example, a nucleic acid construct of the invention. When an expression cassette comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). Thus, for example, a polynucleotide encoding a CRISPR-Cas effector protein, a polynucleotide encoding a deaminase, and a polynucleotide comprising a guide nucleic acid comprised in an expression cassette may each be operably associated with a single promoter or one or more of the polynucleotide(s) may be operably associated with separate promoters (e.g., two or three promoters in any combination), which may be the same or different from each other.

In some embodiments, an expression cassette comprising the polynucleotides/nucleic acid constructs of the invention may be optimized for expression in an organism (e.g., an animal, a plant, a bacterium and the like).

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to a gene encoding a CRISPR-Cas effector protein or a gene encoding a deaminase, may be native to a host cell, or may be native to another source (e.g., foreign or heterologous to the promoter, to a gene encoding the CRISPR-Cas effector protein or a gene encoding the deaminase, to a host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

The expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g., autonomous replicating plasmid with an origin of replication). Additionally, included are shuttle vectors by which is meant a DNA vehicle capable, naturally or by design, of replication in two different host organisms, which may be selected from actinomycetes and related species, bacteria and eukaryotic (e.g., higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid construct of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). Thus, for example, a target nucleic acid may be contacted with a nucleic acid construct of the invention encoding, for example, a nucleic acid binding polypeptide (e.g., a DNA binding domain such as a sequence-specific DNA binding protein (e.g., a polynucleotide-guided endonuclease, a CRISPR-Cas effector protein (e.g., CRISPR-Cas endonuclease), a zinc finger nuclease, a transcription activator-like effector nuclease (TALEN) and/or an Argonaute protein)), a guide nucleic acid, and/or a deaminase under conditions whereby the nucleic acid binding polypeptide is expressed, and the nucleic acid binding polypeptide (e.g., CRISPR-Cas effector protein) forms a complex with the guide nucleic acid, the complex hybridizes to the target nucleic acid, and optionally the deaminase is recruited to the nucleic acid binding polypeptide (and thus, to the target nucleic acid) or the deaminase is fused to the nucleic acid binding polypeptide, thereby modifying the target nucleic acid. In some embodiments, a CRISPR-Cas effector protein, a guide nucleic acid, and a deaminase contact a target nucleic acid to thereby modify the nucleic acid. In some embodiments, the CRISPR-Cas effector protein, a guide nucleic acid, and/or a deaminase may be in the form of a complex (e.g., a ribonucleoprotein such as an assembled ribonucleoprotein complex) and the complex contacts the target nucleic acid. In some embodiments, the complex or a component thereof (e.g., the guide nucleic acid) hybridizes to the target nucleic acid and thereby the target nucleic acid is modified (e.g., via action of the CRISPR-Cas effector protein and/or deaminase). In some embodiments, the deaminase and the nucleic acid binding polypeptide localize at the target nucleic acid, optionally through covalent and/or non-covalent interactions.

As used herein, “modifying” or “modification” in reference to a target nucleic acid includes editing (e.g., mutating), covalent modification, exchanging/substituting nucleic acids/nucleotide bases, deleting, cleaving, and/or nicking of a target nucleic acid to thereby provide a modified nucleic acid and/or altering transcriptional control of a target nucleic acid to thereby provide a modified nucleic acid. In some embodiments, a modification may include an insertion and/or deletion of any size and/or a single base change (SNP) of any type. In some embodiments, a modification comprises a SNP. In some embodiments, a modification comprises exchanging and/or substituting one or more (e.g., 1, 2, 3, 4, 5, or more) nucleotides. In some embodiments, an insertion or deletion may be about 1 base to about 30,000 consecutive bases in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97,98,99,100,110,120, 130,140, 150, 160, 170, 180, 190,200,210,220,230,240,250,260, 270, 280, 290, 300, 310, 320, 330, 340, 350, 360, 370, 380, 390, 400, 410, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, 30,000 consecutive bases in length or more, or any value or range therein). Thus, in some embodiments, an insertion or deletion may be about 1, 2, 3, 4, 5,6,7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300 consecutive bases to about 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 consecutive bases in length, or any range or value therein; about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300 consecutive bases to about 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000 consecutive bases or more in length, or any value or range therein; about 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000 consecutive bases to about 2500, 3000, 3500, 4000, 4500, 5000, 5500, 6000, 6500, 7000, 7500, 8000, 8500, 9000, 9500, or 10,000 consecutive bases or more in length, or any value or range therein; or about 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, or 700 consecutive bases to about 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2500, 3000, 3500, 4000, 4500, or 5000 consecutive bases or more in length, or any value or range therein. In some embodiments, an insertion or deletion may be about 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, or 10,000 consecutive bases to about 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, or 30,000 consecutive bases or more in length, or any value or range therein.

“Recruit,” “recruiting” or “recruitment” as used herein refer to attracting one or more polypeptide(s) or polynucleotide(s) to another polypeptide or polynucleotide (e.g., to a particular location in a genome) using protein-protein interactions, nucleic acid-protein interactions (e.g., RNA-protein interactions), and/or chemical interactions. Protein-protein interactions can include, but are not limited to, peptide tags (epitopes, multimerized epitopes) and corresponding affinity polypeptides, RNA recruiting motifs and corresponding affinity polypeptides, and/or chemical interactions. Example chemical interactions that may be useful with polypeptides and polynucleotides for the purpose of recruitment can include, but are not limited to, rapamycin-inducible dimerization of FRB—FKBP; Biotin-streptavidin interaction; SNAP tag (Hussain et al. Curr Pharm Des. 19(30):5437-42 (2013)); Halo tag (Los et al. ACS Chem Biol. 3(6):373-82 (2008)); CLIP tag (Gautier et al. Chemistry & Biology 15:128-136 (2008)); DmrA-DmrC heterodimer induced by a compound (Tak et al. Nat Methods 14(12):1163-1166 (2017)); Bifunctional ligand approaches (fuse two protein-binding chemicals together) (Voβ et al. Curr Opin Chemical Biology 28:194-201 (2015)) (e.g. dihyrofolate reductase (DHFR) (Kopyteck et al. Cell Cehm Biol 7(5):313-321 (2000)).

“Introducing,” “introduce,” “introduced” (and grammatical variations thereof) in the context of a polynucleotide means presenting a nucleotide sequence of interest (e.g., polynucleotide, a nucleic acid construct, and/or a guide nucleic acid) to a host organism or cell of said organism (e.g., host cell; e.g., a plant cell) in such a manner that the nucleotide sequence gains access to the interior of a cell. Thus, for example, a polynucleotide from an Agrobacterium rhizogenes strain (that may encode or comprise at least a portion of an editing system) may be introduced into a cell of an organism, thereby transforming the cell with the polynucleotide. In some embodiments, a nucleic acid construct of the invention encoding a CRISPR-Cas effector protein, a guide nucleic acid, and a deaminase (e.g., a cytosine deaminase and/or adenine deaminase) may be introduced into a cell of an organism, thereby transforming the cell with the CRISPR-Cas effector protein, guide nucleic acid, and deaminase. In some embodiments, the organism is a eukaryote (e.g., a mammal such as a human).

The term “transformation” as used herein refers to the introduction of a heterologous nucleic acid into a cell. Transformation of a cell may be stable or transient. Thus, in some embodiments, a host cell or host organism may be stably transformed with a polynucleotide/nucleic acid molecule of the invention. In some embodiments, a host cell or host organism may be transiently transformed with a nucleic acid construct of the invention.

“Transient transformation” in the context of a polynucleotide means that a polynucleotide is introduced into a cell (e.g., by a transformation and/or transfection approach) and does not integrate into the genome of the cell; thus, the cell is transiently transformed with the polynucleotide. A nucleic acid that is “transiently expressed” as used herein refers to a nucleic acid that has been introduced into a cell and the nucleic acid is not integrated into the genome of the cell, thereby the cell is transiently transformed with the nucleic acid.

By “stably introducing” or “stably introduced” in the context of a polynucleotide introduced into a cell (e.g., by a transformation and/or transfection approach) is intended that the introduced polynucleotide is stably incorporated into the genome of the cell, and thus the cell is stably transformed with the polynucleotide. A nucleic acid that is “stably expressed” as used herein refers to a nucleic acid that has been introduced into a cell and the nucleic acid is integrated into the genome of the cell, thereby the cell is stably transformed with the nucleic acid.

“Stable transformation” or “stably transformed” as used herein means that a nucleic acid molecule is introduced into a cell (e.g., by a transformation and/or transfection approach) and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

An “edited cell,” “edited plant,” “edited plant part,” “edited root,” “edited callus,” “edited plantlet,” and/or the like as used herein refer to a cell, plant, plant part, root, callus, plant part, and/or the like, respectively, that comprises a modified nucleic acid in that a target nucleic acid been modified using an editing system as described herein to provide the modified nucleic acid. Thus, an “edited cell,” “edited plant,” “edited plant part,” “edited root,” “edited callus,” “edited plantlet,” and/or the like comprise a nucleic acid (i.e., a modified nucleic acid) that has been modified and/or changed compared to its unmodified or native sequence and/or structure.

The terms “transgene” or “transgenic” as used herein refer to at least one nucleic acid sequence that is taken from the genome of one organism, or produced synthetically, and which is then introduced into a host cell (e.g., a plant cell) or organism or tissue of interest and which is subsequently integrated into the host's genome by means of “stable” transformation or transfection approaches. In contrast, the term “transient” transformation or transfection or introduction refers to a way of introducing molecular tools including at least one nucleic acid (DNA, RNA, single-stranded or double-stranded or a mixture thereof) and/or at least one amino acid sequence, optionally comprising suitable chemical or biological agents, to achieve a transfer into at least one compartment of interest of a cell, including, but not restricted to, the cytoplasm, an organelle, including the nucleus, a mitochondrion, a vacuole, a chloroplast, or into a membrane, resulting in transcription and/or translation and/or association and/or activity of the at least one molecule introduced without achieving a stable integration or incorporation and thus inheritance of the respective at least one molecule introduced into the genome of a cell. The term “transgene-free” refers to a condition in which a transgene is not present or found in the genome of a host cell or tissue or organism of interest.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention may be transiently introduced into a cell with a guide nucleic acid and as such, no foreign or introduced DNA is inserted into the genome of the cell.

A nucleic acid construct of the invention can be introduced into a cell by any method known to those of skill in the art. In some embodiments, transformation methods include, but are not limited to, transformation via bacterial-mediated nucleic acid delivery (e.g., via Agrobacteria), viral-mediated nucleic acid delivery, silicon carbide and/or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. In some embodiments of the invention, transformation of a cell comprises nuclear transformation. In some embodiments, transformation of a cell comprises plastid transformation (e.g., chloroplast transformation). In some embodiments, a recombinant nucleic acid construct of the invention can be introduced into a cell via conventional breeding techniques.

Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

A polynucleotide and/or polypeptide can be introduced into a host organism or its cell (optionally a plant, plant part, and/or plant cell) in any number of ways that are well known in the art. The methods of the invention do not depend on a particular method for introducing one or more nucleotide sequences into the organism, only that they gain access to the interior of at least one cell of the organism. Where more than one nucleotide sequence is to be introduced, they can be assembled as part of a single nucleic acid construct, or as separate nucleic acid constructs, and can be located on the same or different nucleic acid constructs. A polynucleotide and/or polypeptide can be introduced into the cell of interest in a single transformation event, and/or in separate transformation events, or, alternatively, a polynucleotide and/or polypeptide can be incorporated into a plant, for example, as part of a breeding protocol. In some embodiments, the cell is a eukaryotic cell (e.g., a mammalian such as a human cell or a plant cell).

As used herein, a “CRISPR-Cas effector protein” is a protein or polypeptide or domain thereof that cleaves, cuts, or nicks a nucleic acid, binds a nucleic acid (e.g., a target nucleic acid and/or a guide nucleic acid), and/or that identifies, recognizes, or binds a guide nucleic acid as defined herein. In some embodiments, a CRISPR-Cas effector protein may be an enzyme (e.g., a nuclease, endonuclease, nickase, etc.) or portion thereof and/or may function as an enzyme. In some embodiments, a CRISPR-Cas effector protein refers to a CRISPR-Cas nuclease polypeptide or domain thereof that comprises nuclease activity or in which the nuclease activity has been reduced or eliminated, and/or comprises nickase activity or in which the nickase has been reduced or eliminated, and/or comprises single stranded DNA cleavage activity (ss DNAse activity) or in which the ss DNAse activity has been reduced or eliminated, and/or comprises self-processing RNAse activity or in which the self-processing RNAse activity has been reduced or eliminated. A CRISPR-Cas effector protein may bind to a target nucleic acid. A CRISPR-Cas effector protein may be a Type I, II, III, IV, V, or VI CRISPR-Cas effector protein. In some embodiments, a CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be a Type II CRISPR-Cas effector protein, for example, a Cas9 effector protein. In some embodiments, a CRISPR-Cas effector protein may be Type V CRISPR-Cas effector protein, for example, a Cas12 effector protein.

In some embodiments, a CRISPR-Cas effector protein may be or include, but is not limited to, a Cas9, C2c1, C2c3, Cas12a (also referred to as Cpf1), Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csx14, Csx10, Csx16, CsaX, Csx3, Csxl, Csx15, Csfl, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site (e.g., RuvC, HNH, e.g., RuvC site of a Cas12a nuclease domain; e.g., RuvC site and/or HNH site of a Cas9 nuclease domain). A CRISPR-Cas effector protein having a mutation in its nuclease active site, and therefore, no longer comprising nuclease activity, is commonly referred to as “dead,” e.g., dCas9. In some embodiments, a CRISPR-Cas effector protein domain or polypeptide having a mutation in its nuclease active site may have impaired activity or reduced activity as compared to the same CRISPR-Cas effector protein without the mutation, e.g., a nickase, e.g, Cas9 nickase, Cas12a nickase.

A CRISPR Cas9 effector protein or CRISPR Cas9 effector domain useful with this invention may be any known or later identified Cas9 nuclease. In some embodiments, a CRISPR Cas9 polypeptide can be a Cas9 polypeptide from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. In some embodiments, a CRISPR-Cas effector protein may be a Cas9 polypeptide or domain thereof and optionally may have a nucleotide sequence of any one of SEQ ID NOs:3-13 or 60-63 and/or an amino acid sequence of any one of SEQ ID NOs:14-15.

In some embodiments, the CRISPR-Cas effector protein may be a Cas9 polypeptide derived from Streptococcus pyogenes and recognizes the PAM sequence motif NGG, NAG, NGA (Mali et al, Science 2013; 339(6121): 823-826). In some embodiments, the CRISPR-Cas effector protein may be a Cas9 polypeptide derived from Streptococcus thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the CRISPR-Cas effector protein may be a Cas9 polypeptide derived from Streptococcus mutans and recognizes the PAM sequence motif NGG and/or NAAR (R=A or G) (See, e.g., Deveau et al, J BACTERIOL 2008; 190(4): 1390-1400). In some embodiments, the CRISPR-Cas effector protein may be a Cas9 polypeptide derived from Streptococcus aureus and recognizes the PAM sequence motif NNGRR (R=A or G). In some embodiments, the CRISPR-Cas effector protein may be a Cas9 protein derived from S. aureus, which recognizes the PAM sequence motif N GRRT (R=A or G). In some embodiments, the CRISPR-Cas effector protein may be a Cas9 polypeptide derived from S. aureus, which recognizes the PAM sequence motif N GRRV (R=A or G). In some embodiments, the CRISPR-Cas effector protein may be a Cas9 polypeptide that is derived from Neisseria meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the CRISPR-Cas effector protein may be a Cas13a protein derived from Leptotrichia shahii, which recognizes a protospacer flanking sequence (PFS) (or RNA PAM (rPAM)) sequence motif of a single 3′ A, U, or C, which may be located within the target nucleic acid.

A Type V CRISPR-Cas effector protein useful with embodiments of the invention may be any Type V CRISPR-Cas nuclease. A Type V CRISPR-Cas nuclease useful with this invention as an effector protein can include, but is not limited, to Cas12a (Cpf1), Cas12b, Cas12c (C2c3), Cas12d (CasY), Cas12e (CasX), Cas12g, Cas12h, Cas12i, C2c1, C2c4, C2c5, C2c8, C2c9, C2c10, Cas14a, Cas14b, and/or Cas14c nuclease. In some embodiments, a Type V CRISPR-Cas nuclease polypeptide or domain useful with embodiments of the invention may be a Cas12a polypeptide or domain. In some embodiments, a Type V CRISPR-Cas effector protein or domain useful with embodiments of the invention may be a nickase, optionally, a Cas12a nickase. In some embodiments, a CRISPR-Cas effector protein may be a Cas12a polypeptide or domain thereof and optionally may have an amino acid sequence of any one of SEQ ID NOs:16-32 and/or a nucleotide sequence of any one of SEQ ID NOs:33-35.

In some embodiments, the CRISPR-Cas effector protein may be derived from Cas12a, which is a Type V Clustered Regularly Interspaced Short Palindromic Repeats (CRISPR)-Cas nuclease. Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein/domain useful with this invention may be any known or later identified Cas12a polypeptide (previously known as Cpf1) (see, e.g., U.S. Pat. No. 9,790,490, which is incorporated by reference for its disclosures of Cpf1 (Cas12a) sequences). The term “Cas12a”, “Cas12a polypeptide” or “Cas12a domain” refers to an RNA-guided nuclease comprising a Cas12a polypeptide, or a fragment thereof, which comprises the guide nucleic acid binding domain of Cas12a and/or an active, inactive, or partially active DNA cleavage domain of Cas12a. In some embodiments, a Cas12a useful with the invention may comprise a mutation in the nuclease active site (e.g., RuvC site of the Cas12a domain). A Cas12a domain or Cas12a polypeptide having a mutation in its nuclease active site, and therefore, no longer comprising nuclease activity, is commonly referred to as deadCas12a (e.g., dCas12a). In some embodiments, a Cas12a domain or Cas12a polypeptide having a mutation in its nuclease active site may have impaired activity, e.g., may have nickase activity.

In some embodiments, a CRISPR-Cas effector protein may be optimized for expression in an organism, for example, in an animal (e.g., a mammal such as a human), a plant, a fungus, an archaeon, or a bacterium. In some embodiments, a CRISPR-Cas effector protein (e.g., Cas12a polypeptide/domain or a Cas9 polypeptide/domain) may be optimized for expression in a plant.

Any deaminase domain/polypeptide useful for base editing may be used with this invention. A “cytosine deaminase” and “cytidine deaminase” as used herein refer to a polypeptide or domain thereof that catalyzes or is capable of catalyzing cytosine deamination in that the polypeptide or domain catalyzes or is capable of catalyzing the removal of an amine group from a cytosine base. Thus, a cytosine deaminase may result in conversion of cystosine to a thymidine (through a uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G→A conversion in antisense (e.g., “−”, complementary) strand of the target nucleic acid. In some embodiments, a cytosine deaminase encoded by a polynucleotide of the invention generates a C to T, G, or A conversion in the complementary strand in the genome.

A cytosine deaminase useful with this invention may be any known or later identified cytosine deaminase from any organism (see, e.g., U.S. Pat. No. 10,167,457 and Thuronyi et al. Nat. Biotechnol. 37:1070-1079 (2019), each of which is incorporated by reference herein for its disclosure of cytosine deaminases). Cytosine deaminases can catalyze the hydrolytic deamination of cytidine or deoxycytidine to uridine or deoxyuridine, respectively. Thus, in some embodiments, a deaminase or deaminase domain useful with this invention may be a cytidine deaminase domain, catalyzing the hydrolytic deamination of cytosine to uracil. In some embodiments, a cytosine deaminase may be a variant of a naturally-occurring cytosine deaminase, including, but not limited to, a primate (e.g., a human, monkey, chimpanzee, gorilla), a dog, a cow, a rat or a mouse. Thus, in some embodiments, a cytosine deaminase useful with the invention may be about 70% to about 100% identical to a wild-type cytosine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring cytosine deaminase).

In some embodiments, a cytosine deaminase useful with the invention may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an atCDA1 (e.g., At2g19570), and evolved versions of the same. Evolved deaminases are disclosed in, for example, U.S. Pat. No. 10,113,163, Gaudelli et al. Nature 551(7681):464-471 (2017)) and Thuronyi et al. (Nature Biotechnology 37: 1070-1079 (2019)), each of which are incorporated by reference herein for their disclosure of deaminases and evolved deaminases. In some embodiments, the cytosine deaminase may be an APOBEC1 deaminase having the amino acid sequence of SEQ ID NO:36. In some embodiments, the cytosine deaminase may be an APOBEC3A deaminase having the amino acid sequence of SEQ ID NO:37. In some embodiments, the cytosine deaminase may be an CDA1 deaminase, optionally a CDA1 having the amino acid sequence of SEQ ID NO:38. In some embodiments, the cytosine deaminase may be a FERNY deaminase, optionally a FERNY having the amino acid sequence of SEQ ID NO:39. In some embodiments, the cytosine deaminase may be a rAPOBEC1 deaminase, optionally a rAPOBEC1 deaminase having the amino acid sequence of SEQ ID NO:40. In some embodiments, the cytosine deaminase may be a hAID deaminase, optionally a hAID having the amino acid sequence of SEQ ID NO:41 or SEQ ID NO:42. In some embodiments, a cytosine deaminase useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical) to the amino acid sequence of a naturally occurring cytosine deaminase (e.g., “evolved deaminases”) (see, e.g., SEQ ID NO:43, SEQ ID NO:44, SEQ ID NO:45). In some embodiments, a cytosine deaminase useful with the invention may be about 70% to about 99.5% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of any one of SEQ ID NOs:36-45 (e.g., at least 80%, at least 85%, at least 90%, at least 92%, at least 95%, at least 96%, at least 97%, at least 98%, at least 99%, or at least 99.5% identical to the amino acid sequence of any one of SEQ ID NOs: 36-45). In some embodiments, a polynucleotide encoding a cytosine deaminase may be codon optimized for expression in a plant and the codon optimized polypeptide may be about 70% to 99.5% identical to the reference polynucleotide.

An “adenine deaminase” and “adenosine deaminase” as used herein refer to a polypeptide or domain thereof that catalyzes or is capable of catalyzing the hydrolytic deamination (e.g., removal of an amine group from adenine) of adenine or adenosine. In some embodiments, an adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid. An adenine deaminase useful with this invention may be any known or later identified adenine deaminase from any organism (see, e.g., U.S. Pat. No. 10,113,163, which is incorporated by reference herein for its disclosure of adenine deaminases).

In some embodiments, an adenosine deaminase may be a variant of a naturally-occurring adenine deaminase. Thus, in some embodiments, an adenosine deaminase may be about 70% to 100% identical to a wild-type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in a plant.

In some embodiments, an adenine deaminase domain may be a wild-type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a TadA polypeptide or TadA domain does not comprise an N-terminal methionine. In some embodiments, a wild-type E. coli TadA comprises the amino acid sequence of SEQ ID NO:46. In some embodiments, a mutated/evolved E. coli TadA* comprises the amino acid sequence of SEQ ID NOs:47-50 (e.g., SEQ ID NOs: 47, 48, 49, or 50). In some embodiments, a polynucleotide encoding a TadA/TadA* may be codon optimized for expression in a plant. In some embodiments, an adenine deaminase may comprise all or a portion of an amino acid sequence of any one of SEQ ID NOs:51-56. In some embodiments, an adenine deaminase may comprise all or a portion of an amino acid sequence of any one of SEQ ID NOs:46-56.

The nucleic acid constructs of the invention comprising a CRISPR-Cas effector protein or a fusion protein thereof may be used in combination with a guide nucleic acid (e.g., guide RNA (gRNA), CRISPR array, CRISPR RNA, crRNA), designed to function with the encoded CRISPR-Cas effector protein or domain thereof, to modify a target nucleic acid. A guide nucleic acid useful with this invention may comprise at least one spacer sequence and at least one repeat sequence. The guide nucleic acid is capable of forming a complex with the CRISPR-Cas nuclease domain encoded and expressed by a nucleic acid construct of the invention and the spacer sequence is capable of hybridizing to a target nucleic acid, thereby guiding the complex to the target nucleic acid, wherein the target nucleic acid may be modified (e.g., cleaved or edited) and/or modulated (e.g., modulating transcription) by a deaminase (e.g., a cytosine deaminase and/or adenine deaminase, optionally present in and/or recruited to the complex).

As an example, a nucleic acid construct encoding a Cas9 domain linked to a cytosine deaminase domain (e.g., to provide a fusion protein) may be used in combination with a Cas9 guide nucleic acid to modify a target nucleic acid, wherein the cytosine deaminase domain of the fusion protein deaminates a cytosine base in the target nucleic acid, thereby modifying the target nucleic acid. In a further example, a nucleic acid construct encoding a Cas9 domain linked to an adenine deaminase domain (e.g., to provide a fusion protein) may be used in combination with a Cas9 guide nucleic acid to modify a target nucleic acid, wherein the adenine deaminase domain of the fusion protein deaminates an adenosine base in the target nucleic acid, thereby modifying the target nucleic acid. In some embodiments, a CRISPR-Cas effector protein (e.g., Cas9) is not fused to a cytosine deaminase and/or adenine deaminase, and the cytosine deaminase and/or adenine deaminase may be recruited to the CRISPR-Cas effector protein.

Likewise, a nucleic acid construct encoding a Cas12a domain (or other selected CRISPR-Cas nuclease, e.g., C2c1, C2c3, Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csx14, Csx10, Csx16, CsaX, Csx3, Csxl, Csx15, Csfl, Csf2, Csf3, Csf4 (dinG), and/or Csf5) may be linked to a cytosine deaminase domain or adenine deaminase domain (e.g., to provide fusion protein) and may be used in combination with a Cas12a guide nucleic acid (or the guide nucleic acid for the other selected CRISPR-Cas nuclease) to modify a target nucleic acid, wherein the cytosine deaminase domain or adenine deaminase domain of the fusion protein deaminates a cytosine base or adenosine base, respectively, in the target nucleic acid, thereby modifying the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target nucleic acid (e.g., a target DNA and/or protospacer), and at least one repeat sequence (e.g., a repeat of a Type V Cas12a CRISPR-Cas system, or a fragment or portion thereof, a repeat of a Type II Cas9 CRISPR-Cas system, or fragment thereof; a repeat of a Type V C2c1 CRISPR Cas system, or a fragment thereof, a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csx14, Csx10, Csx16, CsaX, Csx3, Csxl, Csx15, Csfl, Csf2, Csf3, Csf4 (dinG), and/or Csf5, or a fragment thereof), wherein the repeat sequence may be linked to the 5′ end and/or the 3′ end of the spacer sequence. In some embodiments, the guide nucleic acid comprises DNA. In some embodiments, the guide nucleic acid comprises RNA (e.g., is a guide RNA). The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35(Web Server issue):W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein; e.g., about). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild-type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more) to the same region (e.g., 5′ end) of a wild-type CRISPR Cas repeat nucleotide sequence. In some embodiments, a portion of a repeat sequence may comprise a pseudoknot-like structure at its 5′ end (e.g., “handle”).

A “spacer sequence” or “spacer” as used herein refer to a nucleotide sequence that is complementary to a target nucleic acid (e.g., a target DNA and/or a protospacer). In some embodiments, there may be two or more (e.g., 2, 3, 4, or more) different target nucleic acids and one, two, or more (e.g., 1, 2, 3, 4, or more) different spacers for the two or more different target nucleic acids. A single spacer may be configured to hybridize and/or bind to two or more different nucleic acids, or two or more different spacers may have a different sequence and/or each may be configured to hybridize and/or bind to a different nucleic acid. The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target nucleic acid such as to a target nucleic acid that it is intended to bind. Thus, in some embodiments, the spacer sequence can have one, two, three, four, five, or more mismatches as compared to the target nucleic acid, which mismatches can be contiguous or noncontiguous. In some embodiments, the spacer sequence can have about 70% complementarity to a target nucleic acid. In other embodiments, the spacer nucleotide sequence can have about 80% complementarity to a target nucleic acid. In still other embodiments, the spacer nucleotide sequence can have about 85%, 90%, 95%, 96%, 97%, 98%, 99% or 99.5% complementarity, and the like, to a target nucleic acid (protospacer). In some embodiments, the spacer sequence is 100% complementary to a target nucleic acid. A spacer sequence may have a length from about 13 nucleotides to about 30 nucleotides (e.g., 13, 14, 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a target nucleic acid (e.g., protospacer) that is at least about 13 nucleotides to about 30 nucleotides in length. In some embodiments, the spacer is about 20 nucleotides in length. In some embodiments, the spacer is about 21, 22, or 23 nucleotides in length.

In some embodiments, the 5′ region of a spacer sequence of a guide nucleic acid may be fully complementary to a target nucleic acid, while the 3′ region of the spacer may be substantially complementary to the target nucleic acid (such as for a spacer in a Type V CRISPR-Cas system), or the 3′ region of a spacer sequence of a guide nucleic acid may be fully complementary to a target nucleic acid, while the 5′ region of the spacer may be substantially complementary to the target nucleic acid (such as for a spacer in a Type II CRISPR-Cas system), and therefore, the overall complementarity of the spacer sequence to the target nucleic acid may be less than 100%. Thus, for example, in a guide nucleic acid for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target nucleic acid, while the remaining nucleotides in the 3′ region of the spacer sequence are substantially complementary (e.g., at least about 70% complementary) to the target nucleic acid. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target nucleic acid, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target nucleic acid.

As a further example, in a guide nucleic acid for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target nucleic acid, while the remaining nucleotides in the 5′ region of the spacer sequence are substantially complementary (e.g., at least about 70% complementary) to the target nucleic acid. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target nucleic acid, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target nucleic acid.

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or “target region in the genome” refer to a region of an organism's (e.g., a plant's) genome that comprises a sequence that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid as described herein. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome or mammalian (e.g., human) genome). A target region may be selected from any region of at least 13 consecutive nucleotides (e.g., 13, 14, 15, 16, 17, 18, 19, 20, 21, 22, 23, 24, 25, 26, 27, 28, 29, 30 nucleotides, and the like) located immediately adjacent to a PAM sequence.

A “protospacer sequence” refers to the target double stranded DNA and specifically to the portion of the target nucleic acid that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

```
      5′-NNNNNNNNNNNNNNNNNNN-3′ RNA Spacer (SEQ ID NO: 57)
            ||||||||||||||||||||
3′AAANNNNNNNNNNNNNNNNNNN-5′ Target strand (SEQ ID NO: 58)
    ||||
5′TTTNNNNNNNNNNNNNNNNNNN-3′ Non-target strand (SEQ ID NO: 59)
```

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the present invention provides expression cassettes and/or vectors comprising a nucleic acid construct of the invention (e.g., one or more components of an editing system of the invention). In some embodiments, expression cassettes and/or vectors comprising the nucleic acid constructs of the invention and/or one or more guide nucleic acids may be provided. In some embodiments, a nucleic acid construct of the invention encoding at least a portion of an editing system (e.g., a construct comprising a CRISPR-Cas effector protein, a guide nucleic acid and/or a deaminase) may be comprised on the same or on a separate expression cassette or vector from that comprising one or more guide nucleic acid(s). When the nucleic acid construct encoding at least a portion of an editing system is comprised on separate expression cassette(s) or vector(s) from that comprising the guide nucleic acid, a target nucleic acid may be contacted with (e.g., provided with) the expression cassette(s) or vector(s) encoding the at least a portion of an editing system in any order from one another and the guide nucleic acid, e.g., prior to, concurrently with, or after the expression cassette comprising the guide nucleic acid is provided (e.g., contacted with the target nucleic acid).

Provided according to embodiments of the present invention are methods of transforming a plant cell. In some embodiments, a method of transforming a plant cell comprises introducing a polynucleotide from an Agrobacterium rhizogenes strain into the plant cell to provide a transformed plant cell, thereby transforming the plant cell. The polynucleotide may be T-DNA from the Ti plasmid and/or Ri plasmid of the Agrobacterium rhizogenes strain. In some embodiments, the Ti plasmid comprises a non-Agrobacterium rhizogenes origin of replication (i.e., the Ti plasmid comprises an origin of replication that is not native to Agrobacterium rhizogenes). The origin of replication may be from a different Agrobacterium species such as, but not limited to, Agrobacterium tumefaciens. In some embodiments, the transformed plant cell is a transgenic plant cell. Introducing the polynucleotide from the Agrobacterium rhizogenes strain into the plant cell may comprise contacting the plant cell and the Agrobacterium rhizogenes strain. In some embodiments, the plant cell is infected with the Agrobacterium rhizogenes strain. Exemplary methods for introducing an Agrobacterium rhizogenes strain into a plant cell are known in the art and may be used in a method of the present invention. In some embodiments, a plant cell and/or an explant comprising a plant cell is contacted with an Agrobacterium rhizogenes strain, optionally the plant cell and/or explant may be contacted with a composition (e.g., a suspension and/or culture) comprising the Agrobacterium rhizogenes strain. In some embodiments, the plant cell and/or explant is cultured and/or cultivated with the Agrobacterium rhizogenes strain and/or a composition comprising the Agrobacterium rhizogenes strain. For example, a plant donor explant may be wounded with a surgical scalpel and contacted with and/or provided in an Agrobacterium cell suspension, followed by sonication treatment (e.g., for about 1 minute to about 5 or 10 minutes) to create a micro-wounding on the explant tissue. A composition (e.g., a suspension) comprising Agrobacterium rhizogenes cells in a liquid medium may optionally include about 100 micromoles of acetosyringone. In some embodiments, the composition comprising Agrobacterium rhizogenes cells may have an optical density reading of 0.1 to 1.2 at 660 nm. A composition comprising Agrobacterium rhizogenes cells may be contacted to a plant cell (e.g., a plant cell of an explant such as during explant preparation by cutting using a surgical blade). In some embodiments, an explant is treated with sonication for about 1 minute to about 10 minutes when in contact with (e.g., when in) the Agrobacterium rhizogenes composition to introduce micro-wounding to the explant.

In some embodiments, a method of the present invention comprises introducing all or a portion of an editing system into a plant cell. In some embodiments, an editing system (e.g., an editing system including a CRISPR-Cas effector protein and a guide nucleic acid) is assembled in the form of a complex (e.g., a ribonucleoprotein) prior to being introduced into the plant cell and/or is assembled into the complex during and/or after introduction into the plant cell. For example, a method of the present invention may comprise forming a complex (e.g., a ribonucleoprotein) inside a plant cell after introducing the editing system and/or after introducing a polynucleotide encoding all or a portion of the editing system into the plant cell. Exemplary methods for introducing an editing system or a portion thereof into a plant cell are known in the art and may be used in a method of the present invention. In some embodiments, particle bombardment is used to introduce all or a portion of an editing system into a plant cell. In some embodiments, an Agrobacterium rhizogenes strain is used to introduce all or a portion of an editing system into a plant cell.

In some embodiments, the polynucleotide from the Agrobacterium rhizogenes strain that is introduced into the plant cell may encode for or comprise at least a portion of an editing system that is capable of modifying a target nucleic acid in the plant cell to provide a modified nucleic acid. In some embodiments, the Agrobacterium rhizogenes strain comprises a polynucleotide that encodes all or a portion of an editing system. For example, the Agrobacterium rhizogenes strain may comprise a polynucleotide that encodes a nucleic acid binding polypeptide (e.g., a CRISPR-Cas effector protein) and/or a deaminase and/or may include a guide nucleic acid, optionally wherein the nucleic acid binding polypeptide, deaminase and/or guide nucleic acid are present in the same or different polynucleotides. In some embodiments, the Agrobacterium rhizogenes strain includes a polynucleotide that comprises a guide nucleic acid, and optionally a nucleic acid binding polypeptide (e.g., a CRISPR-Cas effector protein) and/or a deaminase are separately introduced (e.g., using particle bombardment) into the plant cell. In some embodiments, the Agrobacterium rhizogenes strain includes a polynucleotide that encodes a ribonucleoprotein and/or a complex of an editing system. In some embodiments, the Agrobacterium rhizogenes strain includes a polynucleotide that codes for or comprises at least a portion (or all) of an editing system that forms a complex (e.g., a ribonucleoprotein) optionally inside a plant cell after being introduced into the plant cell.

A method of the present invention may introduce a polynucleotide from an Agrobacterium rhizogenes strain, a portion or all of an editing system and/or a polynucleotide encoding or comprising a portion or all of an editing system into a plant cell in one or more (e.g., 1, 2, 3, or more) introducing step(s). When there are two or more introducing steps, the two or more introducing steps may be performed separately and may be performed sequentially or concurrently.

In some embodiments, a polynucleotide from an Agrobacterium rhizogenes strain, a portion or all of an editing system and/or a polynucleotide encoding or comprising a portion or all of an editing system is/are introduced into a root-forming competent cell. A “root-forming competent cell” as used herein refers to a cell that has potential and/or capability to form a root upon being transformed. Exemplary root-forming competent cells include, but are not limited to, cambium layer cells in a plant stem. In some embodiments, the plant cell is a cell (e.g., a root-forming competent cell) in an explant such as, but not limited to, a shoot stem nodal explant, shoot tip explant, node explant, shoot node explant, leaf node explant, a shoot meristem explant, axillary shoot, an etiolated shoot, and/or any portion thereof. The explant may be a shoot node explant, optionally a young shoot node explant. A young shoot node explant may be derived and/or obtained from a shoot of a plant (e.g., greenhouse-grown donor plant) and may contain a fully expanded leaf and axillary meristem. In some embodiments, the explant is a shoot meristem explant. In some embodiments, the explant is an etiolated shoot. In some embodiments, the explant is a leaf node explant, optionally a young leaf node explant. In some embodiments, the explant is collected from a plant (e.g., a greenhouse-grown donor plant). In some embodiments, an explant is cultured in vitro to produce one or more axillary shoot(s) and an axillary shoot may be collected and/or contacted with an Agrobacterium rhizogenes strain, optionally wherein the axillary shoot is separated from rest of the explant and the separate axillary shoot is contacted with the Agrobacterium rhizogenes strain. In some embodiments, an explant that is contacted and/or infected with an Agrobacterium rhizogenes stain is a portion of an explant, optionally a portion of an explant that has been cultured in vitro (e.g., on MS agar medium) for a period of time, and the portion is not limited in size. In some embodiments, an explant is wounded and/or cut at one or more (e.g., 1, 2, 3, 4, 5 or more) locations and/or the explant is cut into two or more (e.g., 2, 3, 4, 5, or more) portions and the wounded and/or cut explant is contacted and/or infected with an Agrobacterium rhizogenes stain. A callus (e.g., a transgenic callus) may form at the cut site and/or wound of the explant that is contacted and/or infected with the Agrobacterium rhizogenes stain. A young leaf node is capable of forming a new shoot and leaf from its axillary meristem and is capable of forming a transgenic root upon infection with an Agrobacterium rhizogenes stain in vitro. In some embodiments, an introducing step of the present invention comprises contacting an Agrobacterium rhizogenes strain and an explant comprising a plant cell such that a polynucleotide of the Agrobacterium rhizogenes strain is introduced into the plant cell of the explant.

Embodiments of the present invention may use one or more (e.g., 1, 2, 3, 4, 5, or more) different types of explants as the donor tissues for A. rhizogenes-mediated transformation. An explant (e.g., a transformation donor explant tissue) may be a nodal tissue freshly collected from a greenhouse-grown plant, a pre-cultured nodal tissue prior to Agrobacterium infection, an etiolated seedling, or an axillary shoot derived from an in vitro cultured node tissue. During a co-culturing step and/or a step of contacting a cell of an explant with A. rhizogenes, A. rhizogenes cells may facilitate the transformation of one or more explant cell(s) (also referred to as transformation competent cells). During and/or after the co-cultivation step and/or contacting step, initiation and formation of a callus can occur at a wounding site (e.g., a cut site or location where the explant is/was cut) of the explant, and the callus may be a transgenic callus (e.g., one or more callus cell(s) include a modified nucleic acid). A transgenic callus may develop into a transgenic root, and hence a composite transgenic plant. In some embodiments, a callus tissue (e.g., a callus) is not collected or separated from the remainder of the explant prior to, during, and/or after the transformation and/or contacting step. In some embodiments, the callus tissue (e.g., a callus) is not collected or separated from the remainder of the explant at any point during the process.

Introducing a polynucleotide from an Agrobacterium rhizogenes strain into a plant cell may comprise concurrently introducing transfer DNA (T-DNA) from the tumor-inducing (Ti) plasmid and hairy root-inducing (Ri) plasmid of the Agrobacterium rhizogenes strain. In some embodiments, the T-DNA from the Ti plasmid and the T-DNA from the Ri plasmid are concurrently integrated into the genome of the plant cell to provide a transgenic plant cell. In some embodiments, T-DNA from the Ti plasmid of an Agrobacterium rhizogenes strain (e.g., a Ti plasmid comprising a non-Agrobacterium rhizogenes origin of replication) is introduced into a plant cell and encodes or comprises a polynucleotide of interest (e.g., a polynucleotide encoding or comprising all or a portion of an editing system and/or a reporter gene). In some embodiments, the Agrobacterium rhizogenes strain is a wild-type Agrobacterium rhizogenes strain optionally comprising a polynucleotide of interest and/or a reporter gene. In some embodiments, a method of the present invention produces a transgenic root that comprises T-DNA from the Ti plasmid of an Agrobacterium rhizogenes strain (e.g., a Ti plasmid comprising a non-Agrobacterium rhizogenes origin of replication) and a plant (e.g., a transgenic plant that is optionally an edited plant) may be produced from the transgenic root.

Exemplary Agrobacterium rhizogenes strains include, but are not limited to, Agrobacterium rhizogenes strain 1193, Agrobacterium rhizogenes strain Qual, Agrobacterium rhizogenes strain K599, Agrobacterium rhizogenes strain A4, and/or Agrobacterium rhizogenes strain MSU440. Agrobacterium rhizogenes strain MSU440 may also be referred to as Agrobacterium rhizogenes(str R) MSU440 Ri (agropine type). Agrobacterium rhizogenes strain MSU440 is accessible from the bank of microbes at the National Institute of Genetic Engineering and Biotechnology (NIGEB), Tehran, Iran and/or is commercially available as Cat. No. ACC-124 from Lifeasible, Suite 209, 17 Ramsey Road, Shirley, NY 11967, lifeasible.com/p/2719/msu440-electroporation-competent-cell/. In some embodiments, Agrobacterium rhizogenes strain MSU440 is provided by and/or obtained from the bank of microbes at the National Institute of Genetic Engineering and Biotechnology (NIGEB), Tehran, Iran and/or from Lifeasible, Suite 209, 17 Ramsey Road, Shirley, NY 11967, lifeasible.com/p/2719/msu440-electroporation-competent-cell/, as Cat. No. ACC-124. In some embodiments, the Agrobacterium rhizogenes strain is a disarmed Agrobacterium rhizogenes strain. A “disarmed Agrobacterium rhizogenes strain” as used herein refers to an Agrobacterium rhizogenes strain whose genome has been modified compared to its native sequence and/or structure so that introduction of a polynucleotide from the strain cannot cause and/or provide the hairy root phenotype in a plant cell expressing the polynucleotide. The hairy root phenotype is characterized by short internodes, wrinkled leaves, abundant roots with extensive lateral branching, and/or increased production certain metabolites (see Tepfer 1984 and Shen et al. 1988). In some embodiments, a disarmed Agrobacterium rhizogenes strain is provided by modifying (e.g., deleting) the T-DNA that contains the rol genes in a manner such that the hairy root phenotype is not provided when the strain infects a plant. In some embodiments, a disarmed Agrobacterium rhizogenes strain is provided by deleting at least a portion (or all) of the T-DNA that contains the rol genes so that the strain lacks the T-DNA and/or a functional rol gene. In some embodiments, a disarmed Agrobacterium rhizogenes strain is devoid of T-DNA in its hairy root-inducing (Ri) plasmid and the Ri plasmid comprises trans factors (e.g., vir genes). In some embodiments, both T-DNAs in the Ri plasmid pRIA4 have been removed in a disarmed Agrobacterium rhizogenes strain. In some embodiments, a disarmed Agrobacterium rhizogenes strain is devoid of a rol gene or lacks a functional rol gene (e.g., comprises a nonfuctional rol gene). Exemplary disarmed Agrobacterium rhizogenes strains include, but are not limited to, an Agrobacterium rhizogenes strain 1193, Agrobacterium rhizogenes strain Qual, Agrobacterium rhizogenes strain A4, Agrobacterium rhizogenes strain K599, and/or Agrobacterium rhizogenes strain MSU440 that is modified so that introduction of a polynucleotide from the strain cannot cause and/or provide the hairy root phenotype in a plant cell expressing the polynucleotide to thereby provide a disarmed Agrobacterium rhizogenes strain 1193, a disarmed Agrobacterium rhizogenes strain Qual, a disarmed Agrobacterium rhizogenes strain A4, a disarmed Agrobacterium rhizogenes strain K599 and/or a disarmed Agrobacterium rhizogenes strain MSU440, respectively. In some embodiments, a rol gene (e.g., a functional rol gene) may be present in an Agrobacterium rhizogenes strain and the rol gene may aid in root formation from a plant cell transformed (e.g., infected) with the Agrobacterium rhizogenes strain and/or stimulate plant growth.

In some embodiments, a disarmed Agrobacterium rhizogenes strain of the present invention comprises a deletion in the Ri plasmid, wherein the deletion is in a region of the MSU440 Ri plasmid from base pair 70,000 to base pair 130,000, with the base pair number of the MSU440 Ri plasmid beginning at the start of the origin of replication of the MSU440 Ri plasmid (i.e., the first nucleotide of the origin of replication is base pair 1), or an optimally aligned region thereto for a different Ri plasmid. In some embodiments, the deletion in the region of the MSU440 Ri plasmid or optimally aligned region thereto is a deletion of about 40,000 base pairs to about 60,000 base pairs. In some embodiments, the deletion in the region of the MSU440 Ri plasmid or optimally aligned region thereto is a deletion of about 50,000 base pairs. In some embodiments, the deletion in the region of the MSU440 Ri plasmid or optimally aligned region thereto is in a region having a sequence identity of about 70%, 75%, 80%, 85%, 90%, 95%, 96%, 97%, 98%, 99%, or 100% to SEQ ID NO:67. In some embodiments, the deletion in the region of the MSU440 Ri plasmid or optimally aligned region thereto has a sequence identity of about 70%, 75%, 80%, 85%, 90%, 95%, 96%, 97%, 98%, 99%, or 100% to SEQ ID NO:68 (i.e., the Ri plasmid of the disarmed Agrobacterium rhizogenes strain is devoid of a sequence having a sequence identity of about 70%, 75%, 80%, 85%, 90%, 95%, 96%, 97%, 98%, 99%, or more to SEQ ID NO:68). In some embodiments, the deletion is in the T-DNA of the Ri plasmid that comprises the rol gene for the MSU440 Ri plasmid or the different Ri plasmid.

In some embodiments, a disarmed Agrobacterium rhizogenes strain of the present invention is prepared from Agrobacterium rhizogenes strain MSU440 by deleting about 70%, 75%, 80%, 85%, 90%, 95%, 96%, 97%, 98%, 99%, or 100% of SEQ ID NO:67 in the Ri plasmid of the Agrobacterium rhizogenes strain MSU440. In some embodiments, a disarmed Agrobacterium rhizogenes strain of the present invention is prepared from Agrobacterium rhizogenes strain MSU440 by deleting about 70%, 75%, 80%, 85%, 90%, 95%, 96%, 97%, 98%, 99%, or 100% of SEQ ID NO:68 in the Ri plasmid of the Agrobacterium rhizogenes strain MSU440. In some embodiments, a disarmed Agrobacterium rhizogenes strain of the present invention is prepared from Agrobacterium rhizogenes strain MSU440, wherein the Ri plasmid of the Agrobacterium rhizogenes strain MSU440 has a sequence of SEQ ID NO:69, and the disarmed Agrobacterium rhizogenes strain is prepared by deleting about 70%, 75%, 80%, 85%, 90%, 95%, 96%, 97%, 98%, 99%, or 100% of SEQ ID NO:68 in the Ri plasmid of the Agrobacterium rhizogenes strain MSU440, to provide the disarmed Agrobacterium rhizogenes strain optionally having a Ri plasmid having a sequence having a sequence identity of about 70%, 75%, 80%, 85%, 90%, 95%, 96%, 97%, 98%, 99%, or 100% of SEQ ID NO:70.

A disarmed Agrobacterium rhizogenes strain of the present invention may comprise a Ri plasmid that is devoid of a functional rol gene. In some embodiments, the Ri plasmid of a disarmed Agrobacterium rhizogenes strain of the present invention is devoid of about 70%, 75%, 80%, 85%, 90%, 95%, 96%, 97%, 98%, 99%, or 100% of one or more of SEQ ID NOs:73-77 and/or devoid of about 70%, 75%, 80%, 85%, 90%, 95%, 96%, 97%, 98%, 99%, or 100% of a sequence encoding one or more of SEQ ID NO:78-82. In some embodiments, the Ri plasmid of a disarmed Agrobacterium rhizogenes strain of the present invention is devoid of a rol gene. In some embodiments, the Ri plasmid of a disarmed Agrobacterium rhizogenes strain of the present invention is devoid of the T-DNA that contains the rol gene. In some embodiments, the Ri plasmid of a disarmed Agrobacterium rhizogenes strain of the present invention is devoid of about 70%, 75%, 80%, 85%, 90%, 95%, 96%, 97%, 98%, 99%, or 100% of SEQ ID NO:71 and/or SEQ ID NO:72. In some embodiments, the Ri plasmid of a disarmed Agrobacterium rhizogenes strain of the present invention is devoid of T-DNA.

The Ri plasmid of a disarmed Agrobacterium rhizogenes strain of the present invention may comprise one or more trans factor(s) of the Ri plasmid of the MSU440 Ri plasmid or another Ri plasmid. In some embodiments, the Ri plasmid of a disarmed Agrobacterium rhizogenes strain comprises a vir gene of the MSU440 Ri plasmid or another Ri plasmid. In some embodiments, the Ri plasmid of a disarmed Agrobacterium rhizogenes strain of the present invention comprises an additional sequence compared to the MSU440 Ri plasmid or another Ri plasmid. The additional sequence may have a length of about 100, 500, 1,000, 1,500 or 2,000 base pairs to about 2,500, 3,000, or 3,500 base pairs. In some embodiments, the additional sequence comprises a nucleotide sequence that confers antibiotic resistance (e.g., an antibiotic resistance cassette such as a gentamicin resistance cassette).

In some embodiments, the Ri plasmid of a disarmed Agrobacterium rhizogenes strain of the present invention comprises a sequence having a sequence identity of about 70%, 75%, 80%, 85%, 90%, 95%, 96%, 97%, 98%, 99%, or 100% to SEQ ID NO:65 and/or a sequence having a sequence identity of about 70%, 75%, 80%, 85%, 90%, 95%, 96%, 97%, 98%, 99%, or 100% to SEQ ID NO:66. The Ri plasmid of a disarmed Agrobacterium rhizogenes strain of the present invention may comprise a sequence having a sequence identity of about 70%, 75%, 80%, 85%, 90%, 95%, 96%, 97%, 98%, 99%, or 100% to SEQ ID NO:64. In some embodiments, the Ri plasmid of a disarmed Agrobacterium rhizogenes strain of the present invention comprises a sequence having a sequence identity of about 70%, 75%, 80%, 85%, 90%, 95%, 96%, 97%, 98%, 99%, or 100% to SEQ ID NO:70. In some embodiments, a disarmed Agrobacterium rhizogenes strain of the present invention comprises a tumor-inducing (Ti) plasmid that comprises a non-Agrobacterium rhizogenes origin of replication, optionally wherein the Ti plasmid comprises an Agrobacterium tumefaciens origin of replication. In some embodiments, a disarmed Agrobacterium rhizogenes strain of the present invention is Agrobacterium rhizogenes strain DS101.1. In some embodiments, a disarmed Agrobacterium rhizogenes strain of the present invention is frozen and/or lyophilized.

In some embodiments, a plant cell is stably transformed with a polynucleotide from an Agrobacterium rhizogenes strain, a portion or all of an editing system and/or a polynucleotide encoding or comprising a portion or all of an editing system. In some embodiments, a polynucleotide (e.g., a polynucleotide from an Agrobacterium rhizogenes strain and/or that encodes or comprises a portion or all of an editing system) is stably expressed in a plant cell such that a transgenic plant cell is provided, and stable expression of the polynucleotide in the transgenic plant cell allows and/or provides for a target nucleic acid to be modified in the plant cell and thereby provides a modified nucleic acid.

In some embodiments, a plant cell is transiently transformed with a polynucleotide from an Agrobacterium rhizogenes strain, a portion or all of an editing system and/or a polynucleotide encoding or comprising a portion or all of an editing system. In some embodiments, a polynucleotide (e.g., a polynucleotide from an Agrobacterium rhizogenes strain and/or that encodes or comprises a portion or all of an editing system) is transiently expressed in the plant cell and transient expression of the polynucleotide in the plant cell allows and/or provides for a target nucleic acid to be modified in the plant cell and thereby provide a modified nucleic acid.

A callus and/or root may be formed from a transformed plant cell (e.g., a plant cell comprising a polynucleotide from an Agrobacterium rhizogenes strain, a portion or all of an editing system and/or a polynucleotide encoding or comprising a portion or all of an editing system). In some embodiments, a transformed plant cell comprises a modified nucleic acid. In some embodiments, the callus is a transgenic callus that optionally comprises the modified nucleic acid. A method of the present invention may comprise producing a root (e.g., a hairy root) from the transformed plant cell and the root optionally includes the modified nucleic acid. A “hairy root” as used herein refers to a root having the hairy phenotype that is formed following introduction and expression of the Ri plasmid T-DNA from an Agrobacterium rhizogenes strain in a plant cell. In some embodiments, a method of the present invention comprises inducing root formation (e.g., a hairy root formation) from a transformed plant cell (that optionally comprises a modified nucleic acid) in the absence of an exogenous plant growth substance (e.g., plant growth regulators and/or hormones), a selection agent (e.g., a selection agent for selecting transformed and/or transgenic cells), and/or a root inducing phytohormone. In some embodiments, little or no auxin supplementation is provided to produce a root from the transformed plant cell. In some embodiments, auxin supplementation and/or a selection agent is provided to produce a root from the transformed plant cell. A root (e.g., hairy and/or edited root) may be produced de novo from an explant in that a root may emerge directly from an excised leaf, a stem segment, a nodal meristem, and/or leaf petiole. In some embodiments, a root (e.g., a hairy and/or edited root) may be derived from a shoot tip of an explant. In some embodiments, a method of the present invention comprises converting a shoot meristem of an explant into a root (e.g., a hairy and/or edited root).

In some embodiments, a callus (e.g., a transgenic and/or edited callus) and/or a root (e.g., a transgenic and/or edited root) may be produced according to a method of the present invention at about 2 or 3 weeks to about 4, 5, or 6 weeks from the time a plant cell was initially contacted with an Agrobacterium rhizogenes strain and/or from the time a polynucleotide from the Agrobacterium rhizogenes strain was introduced into the plant cell. In some embodiments, a callus (e.g., a transgenic and/or edited callus) and/or a root (e.g., a transgenic and/or edited root) may be produced according to a method of the present invention at about 2, 3, 4, 5, or 6 weeks from the time a plant cell was initially contacted with an Agrobacterium rhizogenes strain and/or from the time a polynucleotide from the Agrobacterium rhizogenes strain was introduced into the plant cell.

In some embodiments, a method of the present invention provides a transgenic and/or edited plantlet in reduced time compared to a conventional transformation and/or regeneration method (e.g., a method that involves shoot induction from an explant and/or shoot development and root induction to provide a plantlet). In some embodiments, the plantlet provided according to embodiments of the present invention is transgenic and edited, optionally the transgenic and edited plantlet may be a composite plantlet that is edited. In some embodiments, the plantlet provided according to embodiments of the present invention is transgene free and edited (i.e., an edited plantlet that is transgene free). A method of the present invention may provide a transgenic plantlet and/or edited plantlet in about 2 or 3 weeks to about 4, 5, or 6 weeks from the time a plant cell was initially contacted with an Agrobacterium rhizogenes strain to thereby form the plantlet and/or from the time a polynucleotide from the Agrobacterium rhizogenes strain was introduced into the plant cell to thereby form the plantlet. In some embodiments, a transgenic plantlet and/or edited plantlet may be produced according to a method of the present invention at about 2, 3, 4, 5, or 6 weeks from the time a plant cell was initially contacted with an Agrobacterium rhizogenes strain to thereby form the plantlet and/or from the time a polynucleotide from the Agrobacterium rhizogenes strain was introduced into the plant cell to thereby form the plantlet.

In some embodiments, a method of the present invention comprises producing a transgenic root from a transformed plant cell as described herein. The transgenic root may be a transgenic hairy root and/or may comprise a modified nucleic acid. In some embodiments, the transgenic root comprises a polynucleotide from an Agrobacterium rhizogenes strain, a portion or all of an editing system and/or a polynucleotide encoding or comprising a portion or all of an editing system. In some embodiments, the transgenic root comprises T-DNA from the tumor-inducing (Ti) plasmid of the Agrobacterium rhizogenes strain.

A shoot may be produced from a root produced according to embodiments of the present invention. In some embodiments, the shoot is a transgenic shoot (e.g., comprises a polynucleotide from an Agrobacterium rhizogenes strain, a portion or all of an editing system and/or a polynucleotide encoding or comprising a portion or all of an editing system) that optionally comprise cells including a modified nucleic acid. In some embodiments, the shoot comprises cells including a modified nucleic acid. In some embodiments, the shoot is transgene-free and comprises cells including a modified nucleic acid.

A method of the present invention may comprise producing a plant from a root and/or shoot produced according to embodiments of the present invention. In some embodiments, the plant is transgenic (e.g., comprises a polynucleotide from an Agrobacterium rhizogenes strain, a portion or all of an editing system and/or a polynucleotide encoding or comprising a portion or all of an editing system) and/or the plant comprises cells including a modified nucleic acid (i.e., is an edited plant). In some embodiments, the plant is a transgenic plant that is produced from a transgenic root (e.g., a young or mature transgenic root). In some embodiments, a transgenic plant is fully transgenic in that all cells of the plant include an introduced nucleic acid. In some embodiments, the plant is a transgene-free plant that comprises cells including a modified nucleic acid. A transgene-free, edited plant may be produced from a root comprising cells including the modified nucleic acid. In some embodiments, the plant is a transgenic composite plant. A “transgenic composite plant,” “composite transgenic plant,” or “composite plant” as used herein refer to a plant that includes a portion (e.g., one or more root(s)) that is transgenic and another portion that is non-transgenic. In some embodiments, a transgenic composite plant includes transgenic roots and one or more other portion(s) of the plant are non-transgenic. In some embodiments, a transgenic composite plant includes transgenic roots and optionally non-transgenic aerial tissue and/or non-transgenic roots. In some embodiments, a transgenic composite plant is produced from an explant (e.g., a node explant) following introducing a polynucleotide from and/or upon infection by an Agrobacterium rhizogenes strain. In some embodiments, a transgenic plant is produced from a transgenic root and/or transgenic shoot, and the transgenic plant may comprise cells including a modified nucleic acid. In some embodiments, a composite plant is produced from an explant and optionally from a shoot and/or root from the explant, optionally with the shoot being non-transgenic and devoid of cells including a modified nucleic acid (i.e., the shoot is not edited) and the root being transgenic and comprising cells including a modified nucleic acid (i.e., the root is edited). A progeny plant from a composite plant may comprise cells that include a modified nucleic acid; thus, the progeny plant from the composite plant may be an edited plant.

A method of the present invention may comprise screening for a polynucleotide introduced by an Agrobacterium rhizogenes strain, a modified nucleic acid, and/or a given phenotype. In some embodiments, a callus and/or root is screened for a polynucleotide introduced by an Agrobacterium rhizogenes strain, a modified nucleic acid, and/or a given phenotype. In some embodiments, a progeny plant is screened for a polynucleotide introduced by an Agrobacterium rhizogenes strain, a modified nucleic acid, and/or a given phenotype. Screening may comprise phenotyping. In some embodiments, a method of the present invention comprises performing molecular screening such as molecular screening on a callus, root, and/or progeny plant. The molecular screening may be performed before and/or after phenotyping. Methods of screening are known to those of skill in the art and include, but are not limited to, evaluating gene expression levels such as by using quantitative PCR (qPCT) and/or by physical and/or visual evaluation of the phenotype.

In some embodiments, a method of the present invention comprises screening a root and/or callus for cells comprising a modified nucleic acid such as a nucleic acid that is modified responsive to introducing a polynucleotide from an Agrobacterium rhizogenes strain and/or an editing system. In some embodiments, the screened root and/or callus includes a modified nucleic acid, and a plant may be produced from the edited root and/or edited callus. For example, a segment of a root (e.g., a hairy root) may be screened and the remainder of the root may be used to produce a plant (e.g., an edited and/or transgenic plant) from the root. In some embodiments, the plant produced from the edited root and/or edited callus may be an edited plant (i.e., the plant comprises cells including a modified nucleic acid). The produced plant may be a transgenic plant and/or is an edited plant. In some embodiments, the produced plant is non-transgenic and/or is an edited plant. In some embodiments, the produced plant is transgene free and is an edited plant.

In some embodiments, a method of the present invention comprises screening a root and/or callus for a polynucleotide introduced by an Agrobacterium rhizogenes strain that is transiently expressed in one or more cell(s) of the root and/or callus. If the screened root and/or callus transiently expresses the polynucleotide, then, in some embodiments, the root and/or callus that transiently expresses the polynucleotide may be selected and a plant may be produced from the root and/or callus. For example, a segment of a root (e.g., a hairy root) may be screened to determine if the polynucleotide is transiently expressed and the remainder of the root may be used to produce a plant (e.g., an edited plant) from the root. In some embodiments, the root and/or callus that transiently expresses the polynucleotide may comprise cells that include a modified nucleic acid, thereby the root and/or may be an edited root and/or an edited callus, respectively. In some embodiments, the plant produced from the root and/or callus that transiently expresses the polynucleotide may comprise cells that include a modified nucleic acid, thereby the plant may be an edited plant that is transgene free. The polynucleotide from the Agrobacterium rhizogenes strain may encode for or comprise all or a portion of an editing system. In some embodiments, all or a portion of an editing system may be introduced separately from the polynucleotide from the Agrobacterium rhizogenes strain. In some embodiments, the plant produced from the root and/or callus that transiently expresses the polynucleotide from the Agrobacterium rhizogenes strain is non-transgenic and/or is an edited plant.

In some embodiments, a method of the present invention comprises evaluating efficacy (e.g., transformation and/or expression efficiency) of a polynucleotide from an Agrobacterium rhizogenes strain in a cell, plant part, and/or plant. In some embodiments, a method of the present invention comprises evaluating transformation efficiency and/or genome editing efficiency of a polynucleotide from an Agrobacterium rhizogenes strain in a cell, plant part and/or plant. In some embodiments, a method of the present invention comprises evaluating efficacy (e.g., transformation and/or expression efficiency) of an editing system in a cell, plant part and/or plant. In some embodiments, a method of the present invention comprises evaluating transformation efficiency and/or genome editing efficiency of an editing system in a cell, plant part, and/or plant. A method of the present invention may have a transformation efficiency of at least about 1%, 2%, 3%, 4%, 5%, 6%, 7%, 8%, 9%, 10%, or more in a cell, plant part, and/or plant. In some embodiments, a method of the present invention may provide a transformation efficiency (optionally in Rubus (e.g., blackberry) cells/tissue) of at least 4% with a significantly reduced duration in in vitro culture in that the duration for producing the transformed sample (e.g., a plantlet, plant part, etc.) according to a method of the present invention is reduced such as from at least six weeks for conventional methods to about three weeks.

A method of the present invention may be genotype-independent in that the method may introduce a polynucleotide from an Agrobacterium rhizogenes strain and/or an editing system as described herein into a plant cell regardless of the plant cell's genotype for that plant. In some embodiments, a method of the present invention can introduce a polynucleotide from an Agrobacterium rhizogenes strain and/or an editing system as described herein into a plant cell of every genotype for a particular plant. In some embodiments, a method of the present invention is genotype-independent and/or is less selective on the variety the method works on compared to a method not in accordance with the present invention. In some embodiments, a method of the present invention is genotype-independent and/or is successful with a greater number of varieties than compared to a method not in accordance with the present invention.

A method of the present invention may comprise producing an edited plant. A method of producing an edited plant may comprise introducing a polynucleotide from an Agrobacterium rhizogenes strain and/or all or a portion of an editing system into a plant cell, optionally into a plant cell of an explant, to provide an edited cell. In some embodiments, an edited root, an edited callus, and/or edited plant is produced from the edited cell. In some embodiments, a method of producing an edited plant comprises introducing a polynucleotide from an Agrobacterium rhizogenes strain into a plant cell of an explant to provide an edited cell, wherein the polynucleotide encodes or comprises at least a portion of an editing system that is capable of modifying a target nucleic acid in the plant cell to provide a modified nucleic acid and thereby the edited cell. A root (e.g., an edited transgenic root or edited non-transgenic root) may be produced from the edited cell and an edited plant may be produced from the root. In some embodiments, the polynucleotide from an Agrobacterium rhizogenes strain encodes or comprises all of an editing system and the editing system may be encoded or comprised in one or more polynucleotide(s). In some embodiments, the plant cell is a cell (e.g., a root-forming competent cell) in an explant such as, but not limited to, a shoot stem nodal explant, shoot tip explant, node explant, shoot node explant, leaf node explant, etc.

In a method of producing an edited plant, the plant may be a transgenic plant. In some embodiments, the edited plant may be a composite plant, optionally wherein the composite plant comprises a transgenic root and non-transgenic aerial tissue. In some embodiments, the edited plant may be non-transgenic and/or transgene free. In some embodiments, an edited plant may be produced from a transgene free root that comprises cells including a modified nucleic acid. In some embodiments, the edited plant is transgene free.

In some embodiments, a mature transgenic root may be harvested from an edited plant and/or edited explant and a transgenic plant may be produced from the mature transgenic root. A transgenic plant may comprise transgenic cells and non-transgenic cells, optionally in the same plant part or different plant parts. In some embodiments, a transgenic plant is fully transgenic in that all cells of the plant include an introduced nucleic acid and optionally all cells of the plant may be edited (e.g., comprise a modified nucleic acid). In some embodiments, a transgenic plant is a composite plant in that only a portion of the plant is transgenic (e.g., one or more root(s) of the plant are transgenic). In some embodiments, a transgene free root that comprises cells including a modified nucleic acid is harvested from an edited plant and/or edited explant and a transgene free, edited plant may be produced from the transgene free root. For example, the transgene free root may be harvested from a soil grown edited plant, an edited plant grown in vitro, or an edited explant grown in vitro and the transgene free root may be used to produce a transgene free, edited plant.

In some embodiments, a method of transforming a plant cell comprises introducing a polynucleotide from a disarmed Agrobacterium rhizogenes strain MSU440 into the plant cell to provide a transformed plant cell, thereby transforming the plant cell. In some embodiments, the transformed plant cell is a transgenic plant cell. A callus and/or root may be formed from the transformed plant cell and may optionally be a transgenic callus and/or root. The transformed plant cell may be stably transformed with the polynucleotide from the disarmed Agrobacterium rhizogenes strain MSU440, thereby providing a transgenic plant cell. In some embodiments, stable expression of the polynucleotide from the disarmed Agrobacterium rhizogenes strain MSU440 allows and/or provides for a target nucleic acid to be modified in the plant cell and thereby provides a modified nucleic acid. In some embodiments, the transformed plant cell may be transiently transformed with the polynucleotide from the disarmed Agrobacterium rhizogenes strain MSU440. In some embodiments, transient expression of the polynucleotide from the disarmed Agrobacterium rhizogenes strain MSU440 allows and/or provides for a target nucleic acid to be modified in the plant cell and thereby provides a modified nucleic acid. A plant (e.g., an edited plant) may be produced from the transformed plant cell, callus, and/or root as described herein.

According to some embodiments, a method for evaluating transgene function in a plant cell is provided. A method for evaluating transgene function in a plant may comprise introducing a polynucleotide from an Agrobacterium rhizogenes strain into a plant cell to provide a transformed plant cell comprising a transgene; and evaluating the transformed plant cell to determine transgene function. In some embodiments, the polynucleotide encodes or comprises at least a portion of an editing system that is capable of modifying a target nucleic acid in the plant cell to provide a modified nucleic acid. In some embodiments, the evaluating step comprises detecting the presence and/or expression of the polynucleotide from the Agrobacterium rhizogenes strain in the plant cell. In some embodiments, the evaluating step comprises detecting the presence of a hairy root, detecting the expression of a reporter gene, and/or detecting the presence of a transgenic and/or edited callus and/or transgenic and/or edited root. A method for evaluating transgene function in a plant cell may comprise determining whether a target nucleic acid is modified in the transformed plant cell, plant part (e.g., root and/or callus) and/or plant. In some embodiments, a method for evaluating transgene function in a plant cell comprises detecting the presence of a modified nucleic acid in the transformed plant cell, plant part (e.g., root and/or callus) and/or plant. Evaluating the transformed plant cell may be performed at any time after initial contact with an Agrobacterium rhizogenes strain and/or after introduction of a polynucleotide from the Agrobacterium rhizogenes strain into the plant cell. In some embodiments, a transformed plant cell and/or a callus and/or root comprising the transformed plant cell and/or produced from the transformed plant cell is evaluated at about 2 or 3 weeks to about 4, 5, or 6 weeks from the time a plant cell was initially contacted with an Agrobacterium rhizogenes strain and/or from the time a polynucleotide from the Agrobacterium rhizogenes strain was introduced into the plant cell. In some embodiments, a transformed plant cell and/or a callus and/or root comprising the transformed plant cell and/or produced from the transformed plant cell is evaluated at about 2, 3, 4, 5, or 6 weeks from the time a plant cell was initially contacted with an Agrobacterium rhizogenes strain and/or from the time a polynucleotide from the Agrobacterium rhizogenes strain was introduced into the plant cell.

A method of the present invention may comprise introducing a polynucleotide from an Agrobacterium rhizogenes strain and/or an editing system as described herein into a plant cell such as a dicot plant cell. In some embodiments, the plant cell is a blackberry, raspberry (e.g., red raspberry or black raspberry), artic bramble, or cherry plant cell. In some embodiments, the plant cell is from a plant in the Rubus family. Exemplary plants in the Rubus family include, but are not limited to, Rubus allegheniensis, Rubus occidentalis, Rubus odoratus, and Rubus fruticosus.

According to embodiments of the present invention, a highly efficient transformation method can be achieved by using an Agrobacterium rhizogenes strain as described herein. In some embodiments, a method of the present invention utilizes an Agrobacterium rhizogenes strain to introduce (e.g., deliver) T-DNA into cells competent to form roots, optionally wherein the cells are cells of an explant, and the method may comprise regenerating a shoot from a transgenic root (e.g., a transgenic root from the explant). In some embodiments, the plant cell is a plant cell in the Rubus genus. A method of the present invention may have a high efficiency and/or may allow for recovery of a transgenic plant and/or a transgene free, edited plant. In some embodiments, a method of the present invention provides an edited plant that is devoid of T-DNA integration from the Agrobacterium rhizogenes strain into the plant's genome. A plant produced according to embodiments of the present invention (e.g., a transgene-free edited plant) may be screened (e.g., molecularly screened) and identified at an early stage, for example, as early as at the stage of root induction after infection with an Agrobacterium rhizogenes strain (e.g., an Agrobacterium rhizogenes strain carrying a binary plasmid). In some embodiments, a method of the present invention uses an Agrobacterium rhizogenes transformation method as a platform for rapidly assessing transgene function, such as genome editing efficiency, optionally in Rubus tissues.

In some embodiments, a transgene functionality assay method is provided, which may be used to evaluate an editing system and/or genome editing tool. For example, the method may comprise contacting (e.g., infecting) blackberry explants with an Agrobacterium rhizogenes strain (e.g., Agrobacterium rhizogenes strain MSU440) that includes T-DNA encoding or comprising an editing system and producing transgenic calli and/or hairy roots optionally within about 2 weeks to about 4 weeks after contacting the blackberry explants with the Agrobacterium rhizogenes strain. The transgenic calli and/or hairy roots may be used for determining the editing system's efficacy (e.g., the editing system's efficacy in the Rubus plants).

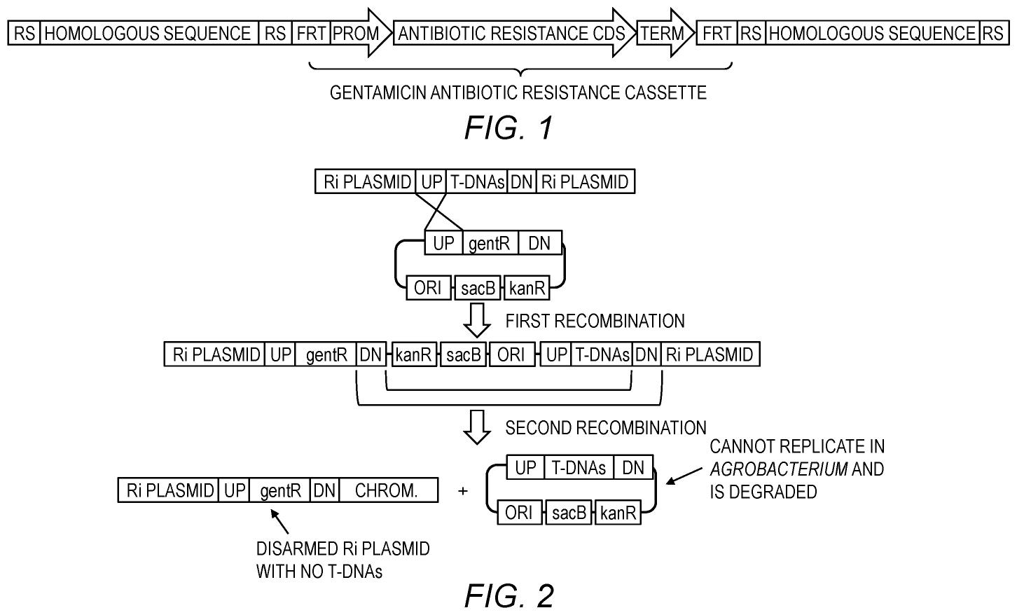

In some embodiments, a method of providing a disarmed Agrobacterium rhizogenes MSU440 is achieved by taking advantage of the endogenous recA. For example, a knockout cassette (e.g., that contains homology arms flanking an antibiotic resistance cassette for an antibiotic (e.g., tetracycline or gentamicin)) as shown, for example, in FIG. 1, is introduced into the Agrobacterium rhizogenes MSU440 cells by electroporation. Post-transformation, the Agrobacterium rhizogenes MSU440 cells are plated in the presence of the antibiotic (e.g., tetracycline or gentamicin). Surviving cells may be screened by colony PCR and/or Sanger sequencing for replacement of the T-DNA with the antibiotic resistance cassette. The method for providing the disarmed Agrobacterium rhizogenes MSU440 may only require the introduction of the knockout cassette since the endogenous recombination system is responsible for homologous recombination.

In some embodiments, a method of providing a disarmed Agrobacterium rhizogenes MSU440 is achieved by homologous recombination mediated by X-red proteins Gam, Bet, and Exo. The X-red recombination system has been used previously to modify the genome of Agrobacterium tumefaciens (see Hu, Shengbiao, et al., Applied microbiology and biotechnology 98.5 (2014): 2165-2172). The method may comprise transforming a plasmid containing an expression cassette for Gam, Bet, and Exo into Agrobacterium rhizogenes MSU440 cells. An antibiotic resistance cassette such as described in regard to FIG. 1 may be transformed into Agrobacterium rhizogenes MSU440 expressing the X-red proteins and plated in the presence of the antibiotic (e.g., tetracycline or gentamicin). Surviving cells may then be screened by colony PCR and/or Sanger sequencing for replacement of the T-DNA with the antibiotic resistance cassette.

In some embodiments, upon verifying T-DNA knockout of an Agrobacterium rhizogenes strain (e.g., Agrobacterium rhizogenes MSU440), the disarmed strain may be used for transformation of a polynucleotide of interest and/or the antibiotic resistance cassette that is in place of the T-DNAs may be removed such as by yeast FLP recombination.

As described herein, the nucleic acids of the invention and/or expression cassettes and/or vectors comprising the same may be codon optimized for expression in an organism. An organism useful with this invention may be any organism or cell thereof for which nucleic acid modification may be useful. An organism can include, but is not limited to, any animal (e.g., mammal), any plant, any fungus, any archaeon, or any bacterium. In some embodiments, the organism may be a plant or cell thereof.

A target nucleic acid of any plant or plant part may be modified using the nucleic acid constructs of the invention. Any plant (or groupings of plants, for example, into a genus or higher order classification) may be modified using the nucleic acid constructs of this invention including an angiosperm, a gymnosperm, a monocot, a dicot, a C3, C4, CAM plant, a bryophyte, a fern and/or fern ally, a microalgae, and/or a macroalgae. A plant and/or plant part useful with this invention may be a plant and/or plant part of any plant species/variety/cultivar. The term “plant part,” as used herein, includes but is not limited to, embryos, pollen, ovules, seeds, leaves, stems, shoots, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. As used herein, “shoot” refers to the above ground parts including the leaves and stems. Further, as used herein, “plant cell” refers to a structural and physiological unit of the plant, which comprises a cell wall and also may refer to a protoplast. A plant cell can be in the form of an isolated single cell or can be a cultured cell or can be a part of a higher-organized unit such as, for example, a plant tissue or a plant organ.

Non-limiting examples of plants useful with the present invention include turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, Chinese cabbage, bok choy), cardoni, carrots, napa, okra, onions, celery, parsley, chick peas, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas, soybeans), an oil plant (rape, canola, mustard, poppy, olive, sunflower, coconut, castor oil plant, cocoa bean, groundnut, oil palm), duckweed, Arabidopsis, a fiber plant (cotton, flax, hemp, jute), Cannabis (e.g., Cannabis sativa, Cannabis indica, and Cannabis ruderalis), lauraceae (cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock. In some embodiments, the nucleic acid constructs of the invention and/or expression cassettes and/or vectors encoding the same may be used to modify maize, soybean, wheat, canola, rice, tomato, pepper, sunflower, raspberry, blackberry, black raspberry and/or cherry. In some embodiments, the plant cell is a blackberry, raspberry (e.g., red raspberry), artic bramble, or cherry plant cell. In some embodiments, the plant cell is from a plant in the Rubus family.

In some embodiments, the invention provides cells (e.g., plant cells, animal cells, bacterial cells, archaeon cells, and the like) comprising one or more polypeptides, polynucleotides, nucleic acid constructs, expression cassettes and/or vectors of the invention.

The present invention further comprises a kit or kits to carry out the methods of this invention. A kit of this invention can comprise reagents, buffers, and apparatus for mixing, measuring, sorting, labeling, etc, as well as instructions and the like as would be appropriate for modifying a target nucleic acid.

In some embodiments, the invention provides a kit for comprising an Agrobacterium rhizogenes strain and/or one or more nucleic acid constructs of the invention, and/or expression cassettes and/or vectors and/or cells comprising the same as described herein, with optional instructions for the use thereof. In some embodiments, a kit may further comprise a CRISPR-Cas guide nucleic acid (corresponding to the CRISPR-Cas effector protein encoded by the polynucleotide of the invention) and/or expression cassettes and/or vectors and or cells comprising the same. In some embodiments, a guide nucleic acid may be provided on the same expression cassette and/or vector as one or more nucleic acid constructs of the invention. In some embodiments, the guide nucleic acid may be provided on a separate expression cassette or vector from that comprising the one or more nucleic acid constructs of the invention.

Accordingly, in some embodiments, kits are provided comprising an Agrobacterium rhizogenes strain and/or a nucleic acid construct comprising (a) a polynucleotide(s) as provided herein and (b) a promoter that drives expression of the polynucleotide(s) of (a). In some embodiments, the kit may further comprise a nucleic acid construct encoding a guide nucleic acid, wherein the construct comprises a cloning site for cloning of a nucleic acid sequence identical or complementary to a target nucleic acid sequence into backbone of the guide nucleic acid.

In some embodiments, the nucleic acid construct of the invention may be an mRNA that may encode one or more introns within the encoded polynucleotide(s). In some embodiments, the nucleic acid constructs of the invention, and/or an expression cassettes and/or vectors comprising the same, may further encode one or more selectable markers useful for identifying transformants (e.g., a nucleic acid encoding an antibiotic resistance gene, herbicide resistance gene, and the like).

A polypeptide, polynucleotide, nucleic acid construct, expression cassette, vector, composition, kit, system and/or cell of the present invention may comprise all or a portion of a sequence of one or more of SEQ ID NOs:1-82. In some embodiments, a polypeptide, polynucleotide, nucleic acid construct, expression cassette, vector, composition, kit, system and/or cell of the present invention may comprise at least about 20%, 25%, 30%, 35%, 40%, 45%, 50%, 55%, 60%, 65%, 70%, 75%, 80%, 85%, 90%, 95%, 96%, 97%, 98%, 99%, or more consecutive amino acids of a sequence of one or more of SEQ ID NOs:1-82.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

Example 1—Comparison of transformation efficiencies in blackberry explants by using Agrobacterium tumefaciens and A. rhizogenes

Transformation experiments were performed to determine T-DNA delivery and the transformation efficiencies by using Agrobacterium tumefaciens or Agrobacterium rhizogenes in the explants. Number of transgenic calli were scored 3-4 weeks after Agrobacterium infection. An Agrobacterium tumefaciens strain pWise1724-pWise1354-AB62 and an Agrobacterium rhizogenes strain pWise1078-MSU440 are used for this experiment. Table 1 provides data from the experiments. Table 1 provides the data from the experiment using blackberry calli as the starting explants. The Agrobacterium rhizogenes strain showed higher transformation efficiency when calli were used as the explants.

TABLE 1
Transformation comparison between Agrobacterium tumefaciens
and Agrobacterium rhizogenes in blackberry. Blackberry calli
initiated from the leaf culture in vitro were used as the transformation
explants. Transgenic calli were selected on medium containing 100
mg/l spectinomycin. The zsGreen positive calli were scored 50 days
after Agrobacterium infection.

| | | | Number of | Trans- |
|---|---|---|---|---|
| | | Number of | zsGreen- | formation |
| | Agrobacterium | Callus | positive | efficiency |
| Construct | strain | Explants | calli | (%) |
| PWise1724 & | A. Tumefaciens - | 223 | 8 | 3.6 |
| pWise1354 | AB62 | | | |
| pWise1078 | A. Rhizogenes- | 96 | 8 | 8.3 |
| | MSU440 | | | |

Transformation experiments were performed to determine transformation efficiencies using Agrobacterium tumefaciens or Agrobacterium rhizogenes in red raspberry etiolated seedlings, similarly to what is described above for blackberry. The Agrobacterium strains all contained the same vector, pWISE3804, which contained the reporter gene, zsGreen. The Agrobacterium strains were Agrobacterium tumefaciens Ab62 and the Agrobacterium rhizogenes strains MSU440, Ara4, Qual and 1193. The efficiency of transformation was scored based on presence of zsGreen in explants 3 days after Agrobacterium infection. All of the Agrobacterium strains were able to transform red raspberry etiolated seedlings to some degree, with Agrobacterium rhizogenes strains Ara4 and MSU440 being the most efficient, followed by Agrobacterium tumefaciens strain Ab62 and Agrobacterium rhizogenes strain Qual, and strain 1193 being the least efficient.

### Example 2

Hairy root induction and generation of a composite plant in blackberry was achieved by transformation using wild-type Agrobacterium rhizogenes strain MSU440 carrying a zsGreen reporter gene from the Ti plasmid. Transformation of blackberry plants was performed essentially as described in Example 4 for the generation of a composite transgenic blackberry plant. The composite transgenic blackberry plant with transgenic roots was evidenced by the expression of the zsGreen fluorescent gene in some of the roots of the composite transgenic blackberry plant and no fluorescence in the aerial part of the plant.

### Example 3

Agrobacterium rhizogenes-mediated transformation in blackberry was used as a rapid editing tool assay. Transgenic calli were generated from blackberry stem explants after infection with Agrobacterium rhizogenes MSU440 strain carrying zsGreen reporter gene and an editing system in its Ti plasmid. The transgenic calli and hairy roots were routinely produced as evidenced by fluorescence of the zsGreen protein. The transgenic calli and hairy roots were sampled for genome editing analysis. In addition, root samples with no detectable fluorescence signals of zsGreen gene were also analyzed for determining gene editing. Similar editing results were obtained from both zsGreen gene-positive samples and zsGreen gene-negative root samples (Table 2).

TABLE 2
Editing assays in blackberry tissues derived from transformation
experiment using the Agrobacterium rhizogenes MSU440 strain.
The transformation construct used was pWise3803 including a
guide RNA for TFL1 gene and a guide RNA for MIXTA gene each
in blackberry. The genotype of blackberry was BK04.

| | | | | Average Editing |
|---|---|---|---|---|
| Type of Tissue | zsGreen | Number of | | Strength (% |
| Sampled | Detected | samples | locus | indels) |
| Callus | Yes | 20 | bk_tfl1 | 67.85 |
| Hairy Roots | Yes | 16 | bk_tfl1 | 68.71 |
| Roots | No | 6 | bk_tfl1 | 74.92 |
| Callus | Yes | 23 | bk_mixta | 24.06 |
| Hairy Roots | Yes | 20 | bk_mixta | 43.09 |
| Roots | No | 8 | bk_mixta | 58.07 |

### Example 4

Composite transgenic plants were produced using the following procedures:

Collected nodal tissue from healthy non-transgenic plants growing in the greenhouse;

Prepare the nodal explants by cutting the nodes into 2-5 cm segments and performing surface-sterilization with 70% ethanol for 1 min and then 20% bleach for 30 min. Nodal explants were then rinsed with sterilized water;

An alternative to the nodal explants was to use etiolated shoots generated from the nodal explants by culturing the nodal explants in the dark and collecting etiolated shoots that formed within the following 3 weeks;

Perform infection of nodal explants or etiolated shoots with an Agrobacterium rhizogenes cell suspension with OD660 readings between 0.5 to 1.0. The strain of A. rhizogenes used includes a T-DNA with at least a marker gene, such as zsGreen, and may also contain a gene(s) of interest.

Co-cultivate the explants (e.g., nodal explants or etiolated shoots) with A. rhizogenes in a humid environment (e.g., 100% humidity), optionally in a closed environment, for 3-5 days;

Wash the explants with sterilized water supplemented with antibiotics;

Transfer and culture the explants on root induction medium without phytohormone for 4-6 weeks during which time new shoots may also start forming;

Identify the transgenic roots by detecting the reporter gene expression (e.g., expression of zsGreen gene) and culture the composite plantlets in growth media to permit continued composite plant development of both roots and aerial plant parts;

Select and culture composite plantlets containing transgenic roots and new axillary shoot in a Plantcon; and

Transplant and grow the composite plantlets in a soil mix by initially planting the composite plantlets in small pots in a growth chamber and then, after further growth, transferring into bigger pots and moving to the greenhouse, thereby providing composite transgenic plants.

### Example 5 Generation of Fully Transgenic Plants from Composite Plants

Fully transgenic plants were produced from mature transgenic hairy roots selected from composite blackberry plants. The marker for transformation was fluorescence of zsGreen which aided in root selection from the composite plant for production of the fully transgenic plant. The transformation experiments were conducted with the A.r. MSU440 strain in blackberry essentially as described in Example 4 for the development of a composite transgenic blackberry plant. The composite plants were grown in the greenhouse for a minimum of 2 weeks and a maximum of 7 weeks. The composite plant was transferred to cold conditions of 5° C., in the dark, for a range of 1 to 6 weeks to produce root induced plants. The aerial portion of the plant was removed and a root was selected from the root mass. Roots that were showing fluorescence of zsGreen were selected, planted into a soil mix and transferred to the greenhouse to be maintained under 16 hours of light at 28 degrees C. with regular watering. A few shoots began to emerge at the 7 day mark after transfer to the greenhouse and many shoots were observed starting at 3 weeks from the transfer to the greenhouse. Phenotype analysis confirmed that the new shoots that had formed expressed zsGreen indicating that the shoots had emerged from transgenic roots and the aerial and root portions of the plant were both transgenic.

### Example 6 Generation of Non-Transgenic, Edited Plants with MSU440

Plant transformation is performed essentially as described in Example 4 except that the A. rhizogenes strain MSU440 is engineered to contain CRISPR editing machinery including a spacer sequence which targets a gene of interest in the plant and a binary vector with ZsGreen gene. A large population of composite plants (approx. 1,000) is generated. The total population of roots in any one of the composite plants will be a range of tissues that are edited, transgenic, and/or hairy or any combination of the foregoing.

The frequency of transgenic but non-hairy roots is determined among the total population of roots generated using wild-type MSU440 containing a binary vector with ZsGreen gene. A molecular assay is used to differentiate hairy roots from non-hairy roots. A molecular assay is also used to determine if the gene of interest is edited by the spacer sequence. Using ZsGreen as a visual marker to distinguish transgenic from non-transgenic roots, combined with the molecular assays, the frequency of transgenic but non-hairy roots among the composite population is determined.

The frequency of transgenic but non-hairy roots may be increased if the frequency was low from the previous experiments. Transformation parameters such as inoculation and co-culture conditions may be used to improve overall transformation efficiency. Hairy root development may be suppressed but non-hairy root formation near the hairy roots may be promoted.

### Example 7 Origin of Replication in the Ti Plasmid Impacts Transformation Efficiency

It has been discovered that the type of the origin of replication in the Ti plasmid can impact the transformation efficiency and the ratio between transgenic roots with or without the rol genes. More than two to three-fold transformation efficiency enhancement is produced when a non-Agrobacterium rhizogenes origin of replication was used in the Ti plasmid (Table 3). It is observed that the number of transgenic foci per explant was also enhanced 2.7-fold due to application of the origin of replication from Agrobacterium tumefaciens (Table 3). Transgenic calli/explants were generated essentially as described in Example 4 except the Agrobacterium rhizogenes strain used contained a Ti plasmid which did not have an Agrobacterium rhizogenes origin of replication as further described below.

TABLE 3
Effect of the origin of replication in the Ti plasmid on the
transformation efficiencies in the blackberry. Two types of
explants, namely the leaf node and shoot stem were infected with
an Agrobacterium rhizogenes strain harboring a construct pWise2717
containing an origin of replication from Agrobacterium rhizogenes
or a construct pWise3792 containing an origin of replication derived
from Agrobacterium tumefaciens. Both of these Agrobacterium
strains contained the reporter gene, zsGreen, which was used to
measure transformation efficiency. The reporter gene zsGreen-positive
transgenic foci on the explants were scored 14 days after Agrobacterium
infection. Transformation efficiency is expressed as the percentage
of explants containing positive zsGreen foci.

| | | | | | AVG zsG+ |
|---|---|---|---|---|---|
| | Explant | | #zsG+ | % TE | Foci/ |
| Construct | type | # Explants | Explants | (Putative) | Explant |
| pWise2717- | Nodes | 33 | 9 | 27.3 | 1.36 |
| MSU | Stem | 65 | 21 | 32.3 | |
| pWise3792- | Nodes | 34 | 31 | 91.2 | 3.7 |
| MSU | Stem | 66 | 53 | 80.3 | |

The effect of the origin replication in the Ti plasmid is further tested with eight constructs, four with the origin of replication from Agrobacterium tumefaciens and another four constructs with the origin of replication from Agrobacterium rhizogenes (Table 4). Significant transformation enhancement was observed by using the origin of replication from Agrobacterium tumefaciens (Table 4).

TABLE 4
Effect of the origin of replication in the Ti plasmid on the transformation
efficiencies in the blackberry. Two types of explants, namely
the leaf node and shoot stem were infected with an
Agrobacterium rhizogenes strain harboring constructs containing an
origin of replication from Agrobacterium rhizogenes or constructs
containing an origin of replication derived from
Agrobacterium tumefaciens. Transformation efficiency is
expressed as the percentage of explants containing positive zsGreen foci.

| | | | | Number of | |
|---|---|---|---|---|---|
| | Source of the | | | zsGreen- | |
| Number of | Origin of | Explant | Number of | positive | |
| Constructs | Replication | type | Explants | Explants | TE (%) |
| 4 | A. tumefaciens | Node | 107 | 78 | 72.9 |
| 4 | A. rhizogenes | Node | 115 | 11 | 9.6 |
| 4 | A. tumefaciens | Stem | 315 | 209 | 66.3 |
| 4 | A. rhizogenes | Stem | 362 | 25 | 6.9 |

Edited roots, non-transgenic, and non-hairy root and composite plants are generated using wild-type MSU440 containing a binary vector with ZsGreen gene and gene editing tools. A large population of composite plants is generated. A visual marker is used to identify the non-transgenic roots from the composite plants. The non-transgenic roots are screened for selecting target gene editing using part of the roots. The edited composite plants are transferred to soil to allow plant growth in order to obtain mature roots for shoot/plant formation. The plants are grown and gene editing in the plant tissue is verified.

### Example 8 Disarmed A. rhizogenes MSU440 Strain DS101.1

The development of a disarmed strain of MSU440 relied on a selectable marker (an antibiotic resistance gene or the counter-selectable marker sacB). Antibiotic susceptibility test on MSU440 were formed with the antibiotics outlined in Table 5.

TABLE 5
Data from the antibiotic susceptibility test.

| antibiotic | concentration | | Resistant or susceptible |
|---|---|---|---|
| streptomycin | 50 | ug/mL | resistant |
| spectinomycin | 50 | ug/mL | resistant |
| carbenicillin | 100 | ug/mL | susceptible |
| kanamycin | 50 | ug/mL | susceptible |
| gentamicin | 35 | ug/mL | susceptible |
| tetracycline | 36 | ug/mL | susceptible |
| chloramphenicol | 30 | ug/mL | resistant |

The sequence of the Ri plasmid in MSU440 was determined and a region of approximately 50 kbp identified for removal from the Ri plasmid to generate the disarmed strain. A homologous recombination knockout cassette was designed as shown in FIG. 1 using gentamicin as the selectable marker. On the inside of the homologous recombination arms, FRT recombination sites (to later remove the antibiotic resistance cassette with FLP recombinase) and a gentamicin resistance cassette (to select for recombinants) were included. The homologous recombination arms were approximately 500-bp directly 5′ and 3′ of the sequence to be knocked out. Disarming MSU440 requires the removal of both T-DNAs and/or the intervening sequence. Once the homologous recombination arms had been identified, the knockout cassette was synthesized and cloned into a binary vector, generating pWISE4108

We first attempted to disarm Agrobacterium rhizogenes MSU440 by transforming in 1 μg of linear, double-stranded knockout cassette DNA PCR-amplified from pWISE4108 and selecting on LB agar plates with streptomycin 50 μg/mL+gentamicin 50 μg/mL. Our attempts at utilizing the recombination proteins in the host organism to facilitate the homologous recombination between the Ri plasmid and the knockout cassette failed—no colonies were observed on plates with the selectable marker for recombination (gentamicin). The first attempt at disarming MSU440 failed so a second strategy using a suicide vector and SacB counterselection was attempted as has been described in the literature (Aliu et al. 2020, Kazuya et al. 2009). The suicide vector and SacB counter-selection strategy works as described below in FIG. 2.

We constructed suicide vector pWISE4502 by replacing the Agrobacterium origin of replication in pWISE4108 with the counter-selectable marker sacB. Five 100 μL aliquots of electrocompetent MSU440 were transformed with 2 μg of pWISE4502 (each). Immediately following electroporation, 900 uL of SOC recovery media was added to each transformation and then incubated at 28° C. with shaking at 200 rpm for 3 hours. At the end of the recovery period, each transformation was plated out onto two LB agar+Strep 50 μg/mL+Kan 50 μg/mL plates (total of 10 plates) to select for single crossover events. The plates were inverted and incubated at 28° C. for approximately 72 hours and then checked for the presence of colonies.

Six colonies were observed on the transformation plates after 72 hours of growth and were named clones DS101.1-DS101.6. Each colony was picked and streaked out to isolation onto LB Agar+Strep 50 μg/mL+Kan 50 μg/mL plates to verify resistance to both streptomycin and kanamycin. Single colonies of each of the six clones were then picked into 4-mL of liquid LB+Strep 50 μg/mL and grown up overnight at 28° C. with 200 rpm shaking. The following day, 100 uL of each overnight culture was plated onto a LB agar+Strep 50 g/mL+5% sucrose plates and incubated at 28° C. for 72 hours to select for the second recombination event to remove sacB.

After 72 hours of growth, each plate had a lawn (entire surface of the plate) of growth. Colonies were streaked out to isolation on LB Agar+Strep 50 μg/mL+5% sucrose and incubated at 28° C. A single colony from each of the six plates was then streaked onto the LB agar plates shown below in Table 6 to determine if the clone should be sequenced to confirm the removal of the approximately 50 kbp region and replacement with a 2.5 kbp region.

TABLE 6
Antibiotic resistances of potentially disarmed Agrobacterium rhizogenes
MSU440 clones. Four clones were moved on to molecular
characterization by colony PCR.

| | Growth on | Growth on | |
|---|---|---|---|
| Clone | Strep + Gent | Strep + Kan | Conclusion |
| DS101.1 | Yes | No | Move on to colony PCR |
| DS101.2 | Yes | Yes | False positive. Eliminate |
| | | | from further testing |
| DS101.3 | Yes | Yes | False positive. Eliminate |
| | | | from further testing |
| DS101.4 | Yes | No | Move on to colony PCR |
| DS101.5 | Yes | No | Move on to colony PCR |
| DS101.6 | Yes | No | Move on to colony PCR |

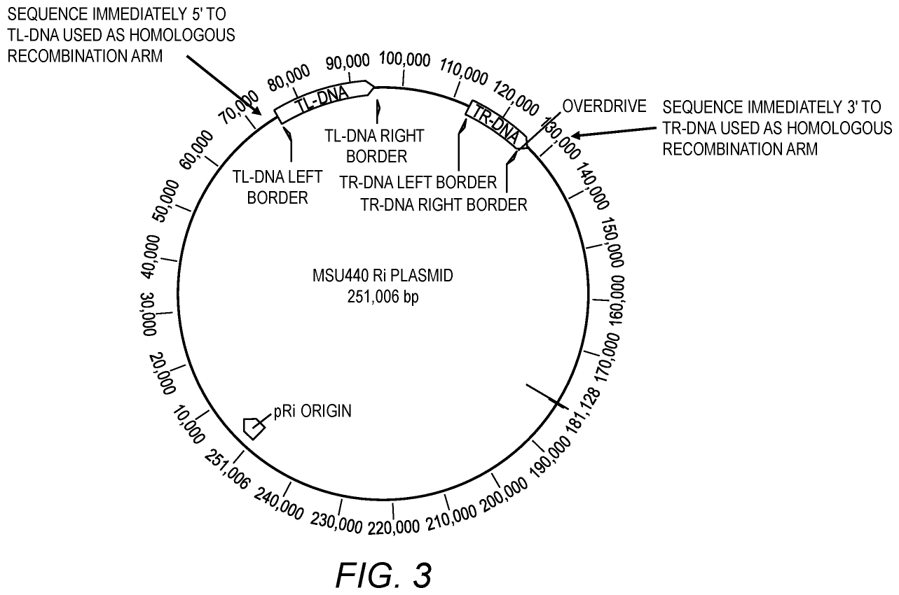

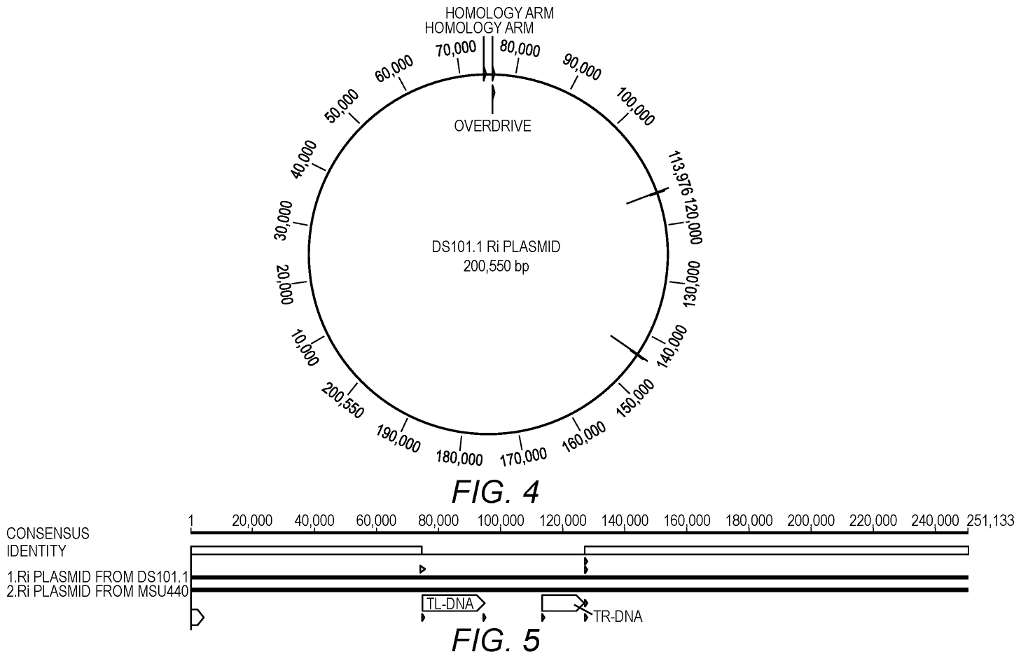

FIG. 3 depicts the Ri plasmid of Agrobacterium rhizogenes MSU440, which includes two T-DNAs that are labeled TL-DNA and TR-DNA, and FIG. 4 depicts the Ri plasmid of Agrobacterium rhizogenes strain DS101.1. Alignment of the Ri plasmid sequence from MSU440 with the Ri plasmid sequence from DS101.1 (FIG. 5) clearly shows the replacement of TL-DNA, TR-DNA and then intervening sequence with the gentamicin resistance cassette, exactly as expected. No presence of TL-DNA, TR-DNA, or the intervening sequence was detected in any other contig from the whole genome sequencing of DS101.1. These sequencing results confirm that we successfully disarmed Agrobacterium rhizogenes MSU440 with the Ri plasmid having a sequence of SEQ ID NO:70. The Ri plasmid sequence of the disarmed Agrobacterium rhizogenes strain DS101.1 (SEQ ID NO:70) includes the 2,000 base pairs upstream of the TL-DNA of MSU440 (SEQ ID NO:65) and 2,000 base pairs downstream of the TR-DNA of MSU440 (SEQ ID NO:66). From the region starting at SEQ ID NO:65 and ending at SEQ ID NO:66, SEQ ID NO:64 of DS101.1, has a percent identity of 64% to SEQ ID NO:67 of MSU440. The disarmed Agrobacterium rhizogenes strain DS101.1 is devoid of the sequence of SEQ ID NO:68 of MSU440.

### REFERENCES

1. Aliu, Ephraim, et al. “Generation of thymidine auxotrophic Agrobacterium tumefaciens strains for plant transformation.” bioRxiv (2020).
2. Kiyokawa, Kazuya, et al. “Construction of disarmed Ti plasmids transferable between Escherichia coli and Agrobacterium species.” Applied and environmental microbiology 75.7 (2009): 1845-1851.

### Example 9—Evaluation of Transformation Efficiencies with an Agrobacterium rhizogenes Strain in Different Cultivars/Genotypes of Blackberry

Transformation experiments in several blackberry genotypes were performed to demonstrate Agrobacterium rhizogenes strain MSU440 transformation of multiple blackberry cultivars. Transformation was performed essentially as described in Example 4 using Agrobacterium rhizogenes strain MSU440 modified to contain a fluorescent gene marker, zsGreen. The results indicate the effectiveness of A. rhizogenes MSU440 strain in T-DNA delivery in different genotypes of Rubus (blackberry) plants (Table 7).

TABLE 7
Transformation tests with Agrobacterium rhizogenes MSU440
strain in three blackberry cultivars. The construct used
in this study was pWise3803. The zsGreen positive transgenic
calli and hairy roots were determined by screening the
cultured explants with a fluorescence scope.

| | | | % Explants |
|---|---|---|---|
| | Number | % Explants with | with zsGreen |
| | of | zsGreen Positive | positive |
| Cultivar | explants | Calli/Hairy Roots | Hairy Roots |
| BK01 | 165 | 69.09 | 1.82 |
| BK04 | 178 | 89.33 | 45.51 |
| BK13 | 202 | 72.77 | 4.95 |

### Example 10—Blackberry Transformation with Disarmed Agrobacterium rhizogenes MSU440 Strain DS101.1

Transformation experiments were conducted to test the disarmed A.r. MSU440 strain, DS101.1 in blackberry. Transformation efficiencies between 62% to 79% were obtained when the wild-type A.r. MSU440 strain harboring construct pWise3880 was used in the transformation method substantially as described in Example 4 except that the root induction media was supplemented with 2,4-D and chlorosulfuron. The rol genes from the A.r. MSU440 strain were removed in the process of developing the DS101.1 strain. The DS101.1 strain maintains its ability to transform plant cells; however, the transgenic plant cells do not display the rol gene's functionality of inducing transgenic calli and hairy roots. This lack of phenotype results in significant reduction in apparent transformation efficiency with absence of auxin and selection agent (Table 8). In the presence of auxin (2,4-D) and chlorosulfuron, the transgenic cells derived from using the disarmed A.r. MSU440 strain DS101.1 also harbored a chlorosulfuron resistant gene developed to visible transgenic calli (Treatment T-9 and T-12, Table 8).

TABLE 8
Transformation evaluation with disarmed
A.r. MSU440 strain DS101.1 in blackberry.

| | | | | % |
|---|---|---|---|---|
| | | | | Explants |
| | | | | with |
| | | | | zsGreen |
| | | Callus Induction | Number of | positive |
| Treatment | p Wise | Media | Explants | Calli |
| T-1 | pWise3880- | ICM | 24 | 79.2 |
| | MSU440 | | | |
| T-2 | pWise3880- | ICM + 1 mg/l 2,4-D + | 24 | 70.8 |
| | MSU440 | 0.2 mg/l BAP | | |
| T-3 | pWise3880- | ICM + 1 mg/l 2,4-D + | 16 | 62.5 |
| | MSU440 | 0.2 mg/l BAP + 0.2 | | |
| | | uM Chloro | | |
| T-4 | pWise3804- | ICM | 12 | 0.0 |
| | DS101.1-1 | | | |
| T-5 | pWise3804- | ICM + 1 mg/l 2,4-D + | 10 | 0.0 |
| | DS101.1-1 | 0.2 mg/l BAP | | |
| T-6 | pWise3804- | ICM + 1 mg/l 2,4-D + | 11 | 0.0 |
| | DS101.1-1 | 0.2 mg/l BAP + 0.2 | | |
| | | uM Chloro | | |
| T-7 | pWise3880- | ICM | 29 | 3.4 |
| | DS101.1-1 | | | |
| T-8 | pWise3880- | ICM + 1 mg/l 2,4-D + | 34 | 5.9 |
| | DS101.1-1 | 0.2 mg/l BAP | | |
| T-9 | pWise3880- | ICM + 1 mg/l 2,4-D + | 12 | 25.0 |
| | DS101.1-1 | 0.2 mg/l BAP + 0.2 | | |
| | | uM Chloro | | |
| T-10 | pWise3880- | ICM | 23 | 0.0 |
| | DS101.1-2 | | | |
| T-11 | pWise3880- | ICM + 1 mg/l 2,4-D + | 48 | 4.2 |
| | DS101.1-2 | 0.2 mg/l BAP | | |
| T-12 | pWise3880- | ICM + 1 mg/l 2,4-D + | 45 | 20.0 |
| | DS101.1-2 | 0.2 mg/l BAP + 0.2 | | |
| | | uM Chloro | | |

(ICM: hairy root induction medium without phytohormone and selection agent; chloro: chlorosulfuron; DS101.1-1 and DS101.1-2: two disarmed MSU440 strains carrying different transformation plasmids; pWise3880: transformation construct containing a chlorosulfuron resistant gene and zsGreen visual marker in its T-DNA; pWise3804: a transformation construct containing zsGreen visual marker gene in its T-DNA)

### Example 11—Generation of Edited Plants with and without T-DNA Integration in Blackberry

Experiments are conducted to test the processes of recovering gene edited Rubus plants which contain and do not contain T-DNA in their genome.

In one experiment, transformation experiments are performed by using wild-type A. rhizogenes strain with Ri and Ti T-DNAs concurrently which are modified to contain the marker gene, zsGreen, as well as genes necessary for editing a target of interest. Composite plants are generated substantially as described in Example 4 and regenerated roots of zsGreen-positive and zsGreen-negative phenotypes after A. rhizogenes infection are collected and screened for detecting gene editing activities, as well as the presence and absence of rol genes and T-DNA of Ti plasmid. Transgenic edited roots show the presence of Ti TDNA (+/−Ri T-DNA) and the editing activities on the target gene. Non-transgenic edited roots show absence of Ri and Ti T-DNA, but the presence of editing activity on the gene target. The selected roots are cultured for recovering the edited plants substantially as described in Example 5.

In another experiment, transformation experiments are performed by using a disarmed A. rhizogenes strain with Ti T-DNA only which is modified to contain a marker gene, zsGreen, as well as genes necessary for editing a target gene. Composite plants are generated substantially as described in Example 4, and regenerated roots of zsGreen-positive and zsGreen-negative phenotypes after A. rhizogenes infection are collected and screened for detecting gene editing activities, and the presence or absence of T-DNA of Ti plasmid. Transgenic edited roots show the presence of Ti TDNA and the editing activities on the target gene. Non-transgenic edited roots show the absence of Ti T-DNA, but the presence of editing activity on the gene target. The selected roots are cultured for recovering the edited plants from the roots substantially as described in Example 5.

In a third experiment, transformation experiments are carried out by using a disarmed A. rhizogenes strain harboring Ti T-DNA which contains two gRNAs targeting to the gene of interest and a herbicide resistance gene such as a modified acetolactate synthase (ALS). Composite plants are generated essentially as described in Example 4 except that after Agrobacterium infection, the explants are subjected to herbicide selection for recovering roots which are tolerant to the corresponding herbicide. A presence or absence molecular assay for the Ti T-DNA in the roots is performed to determine if the roots are transgenic (with the T-DNA) or non-transgenic (without T-DNA). Next generation sequencing analysis is performed to identify editing activities on the target gene (the gene of interest). The selected roots are cultured for recovering the edited plants from the roots substantially as described in Example 5.

### Example 12—Agrobacterium rhizogenes-Mediated Transformation—Strain Evaluation in Black Raspberry and Blackberry

Black raspberry leaf node and stem explants were harvested from the greenhouse, and surface-sterilized with 70% ethanol for 1 min; 20% bleach solution for 30 min; and then rinsed with sterile water 4-5 times. The explants were cultured on MS agar medium for 6-14 days at 25C light. The explants were then cut into 1 to 2 cm segments and contacted with an Agrobacterium cell suspension (OD660=0.6). The explants were incubated in the Agrobacterium cell suspension (e.g., by submerging the explants in the suspension) for about 15 to 30 min. The co-cultivation step was done by transferring the explants to the co-cultivation agar medium with one layer of sterile filter paper and incubating at 23C for 3 days. After the co-cultivation step, explants were washed with sterile water supplemented with antibiotics (250 mg/l Carbenicillin and 200 mg/l Timentin). Explants were then transferred to and cultured on MS medium containing antibiotics (250 mg/l Carbenicillin and 200 mg/l Timentin) under light, at 25C for up to 6 weeks. Evaluation of transformation efficiencies was performed by scoring zsGreen calli or hairy roots with a fluorescence dissection scope.

High transformation efficiencies in the black raspberry were observed by using either Agrobacterium rhizogenes strains A.r.A4 or MSU440 (Table 9). In comparing these two strains, A.r. strain MSU440 not only provides high transformation efficiency, but also promotes rapid growth of the zsGreen-positive transgenic calli and/or hairy roots (Table 9). In contrast, the A.r. strain K599 does not yield transgenic calli in this experiment.

TABLE 9
Transformation evaluation by comparing three different Agrobacterium rhizogenes
strains in black raspberry (Cultivar BR-11). Agrobacterium rhizogenes
strains harboring zsGreen gene in their Ti plasmids (pWise3804) were
compared in the transformation experiment.

| | | | Number | | |
|---|---|---|---|---|---|
| | | | of | % | Size of |
| | | | zsGreen- | zsGreen- | zsGreen- |
| Agrobacterium | | Number of | positive | positive | positive |
| Strain | Construct | Explants | Explants | Explants | Calli |
| A.r. A4 | pWise3804 | 138 | 114 | 82.6 | Small |
| A.r. K599 | pWise3804 | 127 | 0 | 0.0 | N/A |
| A.r. MSU440 | pWise3804 | 128 | 122 | 95.3 | Medium to |
| | | | | | Large |

A similar experiment was also conducted in blackberry. The leaf node and stem explants were harvested from the greenhouse, and surface-sterilized with 70% ethanol for 1 min; 20% bleach solution for 30 min; and then rinsed with sterile water 4-5 times. The explants were cultured on MS agar medium for 8 days at 25C light. Agrobacterium infection was performed by cutting the above prepared explants into 1 to 2 cm segments in the Agrobacterium cell suspension (OD660=0.8-1.0). The explants submerged in the Agrobacterium cell suspension were then sonicated for 2 min, and then incubated for about 15 to 30 min. The co-cultivation step was done by transferring the explants to the co-cultivation agar medium with one layer of sterile filter paper and incubating at 23C for 5 days. After the co-cultivation step, explants were washed with sterile water supplemented with antibiotics (250 mg/l Carbenicillin and 200 mg/l Timentin). Explants were then transferred to and cultured on MS medium containing antibiotics (250 mg/l Carbenicillin and 200 mg/l Timentin) under light, at 25C for up to 6 weeks. Evaluation of transformation efficiencies was performed by scoring zsGreen calli or hairy roots with a fluorescence dissection scope.

High transformation efficiencies in the blackberry were observed by using either Agrobacterium rhizogenes strains A.r.A4 or MSU440 (Table 10). Similarly, A.r. strain MSU440 provides high transformation efficiency and promotes rapid growth of the zsGreen-positive transgenic calli and/or hairy roots (Table 10).

Table 10: Transformation evaluation by comparing two different Agrobacterium rhizogenes strains in blackberry (Cultivar BK-13). Agrobacterium rhizogenes strains harboring zsGreen gene in their Ti plasmids (pWise3804) were used in the transformation experiment.

TABLE 10
Transformation evaluation by comparing two different
Agrobacterium rhizogenes strains in blackberry (Cultivar BK-13).
Agrobacterium rhizogenes strains harboring zsGreen gene in their Ti
plasmids (pWise3804) were used in the transformation experiment.

| | | | Number | |
|---|---|---|---|---|
| | | | of | |
| | | | zsGreen- | % zsGreen- |
| Agrobacterium | | Number of | positive | positive |
| Strains | Construct | Explants | Explants | Explants |
| A.r. A4 | pWise3804 | 31 | 18 | 58.1 |
| A.r. MSU440 | pWise3804 | 15 | 13 | 86.7 |

### Example 13—Black Raspberry and Blackberry Transformation with Agrobacterium rhizogenes Strain MSU440: Transformation Responses by Using Different Types of Explants

Black raspberry leaf node and stem explants were harvested from the greenhouse, and surface-sterilized with 70% ethanol for 1 min; 20% bleach solution for 30 min; and then rinsed with sterile water 4-5 times. The explants were cultured on MS agar medium for 6-14 days at 25C light. After culturing the explants on the MS medium, axillary shoots started to develop from the leaf nodal meristem. The explants were then cut into 1 to 2 cm segments and the newly developed axillary shoots were cut off. The cut explant material and the cut off axillary shoots were contacted with an Agrobacterium cell suspension (OD660=0.6). The explants were incubated in the Agrobacterium cell suspension (e.g., by submerging the explants in the suspension) for about 15 to 30 min. The co-cultivation step was done by transferring the explants to the co-cultivation agar medium with one layer of sterile filter paper and incubating at 23C for 3 days. After the co-cultivation step, explants were washed with sterile water supplemented with antibiotics (250 mg/l Carbenicillin and 200 mg/l Timentin). Explants were then transferred to and cultured on MS medium containing antibiotics (250 mg/l Carbenicillin and 200 mg/l Timentin) under light, at 25C for up to 6 weeks. Evaluation of transformation efficiencies was performed by scoring zsGreen calli or hairy roots with a fluorescence dissection scope.

When transformation explants derived from the greenhouse grown plants were cultured for 6 to 14 days prior to transformation, the newly developed axillary shoots and the nodes/stem segments are both highly transformable with A.r. strain MSU440 in the black raspberry (Table 11) and blackberry (Table 12). Surprisingly, the axillary shoots that had developed in vitro prior to contact with the Agrobacterium cell suspension, were able to form hairy roots easily following contact with the Agrobacterium cell suspension, hence providing composite plants.

TABLE 11
Transformation with node/stem and axillary shoot as the
target explants in Black raspberry. Agrobacterium rhizogenes
strain MSU440 harboring a Ti plasmid with kanamycin resistant
marker NPT-II gene (pWise3737) and the Black raspberry
cultivar BR-11 was used in this experiment

| | | | Number | % |
|---|---|---|---|---|
| | | | of | zsGree |
| | | | zsGreen- | n- |
| Type of | | Number of | positive | positive |
| Explants | Selection | Explants | Explants | Explants |
| Node/Stem | 150 mg/l Kan | 120 | 120 | 100 |
| Axillary Shoot | 150 mg/l Kan | 60 | 60 | 100 |

TABLE 12
Transformation with node/stem and axillary shoot as the target explants
in blackberry (Cultivar BK-13). Transformation data were obtained
by pooling transformation efficiency data from using three transformation
constructs (pWise4847, pWise4961, pWise5043) in
Agrobacterium rhizogenes strain MSU440 in this experiment.

| | | Number of | |
|---|---|---|---|
| | Number | zsGreen- | % zsGreen- |
| Type of | of | positive | positive |
| Explants | Explants | Explants | Explants |
| Node/Stem | 59 | 34 | 57.6 |
| Axillary Shoot | 35 | 29 | 82.9 |

The foregoing is illustrative of the present invention and is not to be construed as limiting thereof. The invention is defined by the following claims, with equivalents of the claims to be included therein.

That which is claimed is:

1. A method of transforming a plant cell, the method comprising:
introducing a polynucleotide from an Agrobacterium rhizogenes strain into the plant cell to provide a transformed plant cell, thereby transforming the plant cell.

2. The method of claim 1, wherein the polynucleotide encodes or comprises at least a portion of an editing system that is capable of modifying a target nucleic acid in the plant cell to provide a modified nucleic acid, optionally wherein the polynucleotide encodes or forms a ribonucleoprotein.

3. The method of claim 1 or 2, wherein the plant cell is a root-forming competent cell.

4. The method of any one of the preceding claims, wherein the introducing step comprises contacting the Agrobacterium rhizogenes strain and an explant (e.g., a shoot stem nodal explant, shoot tip explant, node explant, etc.) comprising the plant cell, optionally wherein the plant cell is present in a callus of the explant and the Agrobacterium rhizogenes strain is contacted to the callus to provide a transformed callus.

5. The method of any one of the preceding claims, wherein introducing the polynucleotide from the Agrobacterium rhizogenes strain into the plant cell comprises concurrently introducing transfer DNA (T-DNA) from a tumor-inducing (Ti) plasmid and hairy root-inducing (Ri) plasmid of the Agrobacterium rhizogenes strain, optionally wherein the T-DNA from the Ti plasmid (e.g., a Ti plasmid comprising a non-Agrobacterium rhizogenes origin of replication) and the T-DNA from the Ri plasmid are concurrently integrated into the genome of the plant cell to provide a transgenic plant cell.

6. The method of any one of the preceding claims, further comprising forming a callus (e.g., a transgenic callus) from the transformed plant cell or culturing the transformed callus.

7. The method of any one of the preceding claims, wherein the plant cell is stably transformed with the polynucleotide.

8. The method of any one of the preceding claims, wherein the polynucleotide is stably expressed in the transformed plant cell and wherein the method further comprises modifying a target nucleic acid in the transformed plant cell to provide a modified nucleic acid.

9. The method of any one of claims 1-6, wherein the plant cell is transiently transformed with the polynucleotide.

10. The method of any one of claims 1-6 or 9, wherein the polynucleotide is transiently expressed in the transformed plant cell and wherein the method further comprises modifying a target nucleic acid in the transformed plant cell to provide a modified nucleic acid.

11. The method of any one of the preceding claims, further comprising producing a root (e.g., a hairy root) from the transformed plant cell or edited plant cell, optionally wherein the root comprises cells including a modified nucleic acid.

12. The method of claim 11, wherein producing the root from the transformed plant cell or edited plant cell comprises inducing root formation in the absence of an exogenous plant growth substance (e.g., plant growth regulators and/or hormones).

13. The method of claim 11 or 12, wherein the root is produced de novo from the explant.

14. The method of claim 11 or 12, wherein the root is derived from a shoot tip of the explant, optionally wherein a shoot meristem explant is converted into a composite plant.

15. The method of any one of the preceding claims, further comprising producing a transgenic root from the transformed plant cell, optionally wherein the transgenic root is a transgenic hairy root.

16. The method of claim 15, wherein the transgenic root comprises the polynucleotide, optionally wherein the polynucleotide comprises transfer DNA (T-DNA) from a plasmid in the Agrobacterium rhizogenes strain (e.g., from a Ti plasmid optionally comprising a non-Agrobacterium rhizogenes origin of replication), optionally wherein the polynucleotide is an engineered recombinant polynucleotide.

17. The method of any one of the preceding claims, further comprising evaluating the efficacy of the polynucleotide, optionally wherein evaluating the efficacy of the polynucleotide comprises evaluating the transformation efficiency and/or genome editing efficiency of the polynucleotide.

18. The method of any one of claims 11-17, further comprising generating a shoot from the root (e.g., a transgenic root), optionally wherein the shoot is transgenic and/or the shoot comprises cells including the modified nucleic acid.

19. The method of claim 18, wherein the shoot is transgene-free and comprises cells including the modified nucleic acid.

20. The method of any one of claims 11-19, further comprising producing a plant from the root and/or shoot, optionally wherein the plant is transgenic and/or the plant comprises cells including the modified nucleic acid.

21. The method of claim 20, wherein the plant is a transgenic plant, optionally wherein the transgenic plant is produced from a mature transgenic root.

22. The method of claim 20, wherein the plant is a transgene-free plant that comprises cells including the modified nucleic acid, optionally wherein the transgene-free plant is produced from a root comprising cells including the modified nucleic acid.

23. The method of claim 20, wherein the plant is a transgenic composite plant, optionally wherein the transgenic composite plant is produced from a node explant upon infection by Agrobacterium rhizogenes strain.

24. The method of claim 23, wherein the transgenic composite plant comprises transgenic roots and optionally non-transgenic aerial tissue and/or non-transgenic roots.

25. The method of any one of the preceding claims, wherein the Agrobacterium rhizogenes strain is a wild-type Agrobacterium rhizogenes strain.

26. The method of any one of claims 1-24, wherein the Agrobacterium rhizogenes strain is a disarmed Agrobacterium rhizogenes strain (i.e., the disarmed Agrobacterium rhizogenes strain is devoid of T-DNA in its hairy root-inducing (Ri) plasmid and the Ri plasmid comprises trans factors (e.g., vir genes).

27. The method of claim 26, wherein the disarmed Agrobacterium rhizogenes strain is a disarmed Agrobacterium rhizogenes strain 1193, a disarmed Agrobacterium rhizogenes strain A4, a disarmed Agrobacterium rhizogenes strain Qual, a disarmed Agrobacterium rhizogenes strain K599 and/or a disarmed Agrobacterium rhizogenes strain MSU440 (e.g., Agrobacterium rhizogenes strain DS101.1 and/or Agrobacterium rhizogenes strain comprising a sequence of SEQ ID NO:70).

28. The method of any one of the preceding claims, wherein the Agrobacterium rhizogenes strain is selected from the group consisting of Agrobacterium rhizogenes strain 1193, Agrobacterium rhizogenes strain A4; Agrobacterium rhizogenes strain Qual, Agrobacterium rhizogenes strain K599 and Agrobacterium rhizogenes strain MSU440.

29. The method of any one of the preceding claims, further comprising screening for the polynucleotide, a modified nucleic acid, or a given phenotype, optionally wherein the screening comprises screening the callus and/or root for the polynucleotide, the modified nucleic acid, or the given phenotype.

30. The method of claim 29, wherein the screening comprises screening the root for the polynucleotide that is transiently expressed in cells of the root and, responsive to the screening, selecting the root that transiently expresses the polynucleotide, and producing a plant from the root that transiently expresses the polynucleotide, wherein the plant comprises cells including the modified nucleic acid.

31. The method of claim 29, wherein the screening comprises screening the root for cells comprising the modified nucleic acid and, responsive to the screening, selecting the root comprising cells including the modified nucleic acid, and producing a plant from the root comprising cells including the modified nucleic acid, wherein the plant comprises cells including the modified nucleic acid.

32. The method of any one of the preceding claims, further comprising performing molecular screening for the polynucleotide, a modified nucleic acid, or a given phenotype, optionally wherein performing molecular screening comprises performing molecular screening on the root for the polynucleotide, the modified nucleic acid, or the given phenotype.

33. The method of any one of the preceding claims, wherein the plant cell is a dicot plant cell, optionally wherein the plant cell is a blackberry, raspberry (e.g., red raspberry), artic bramble, or cherry plant cell.

34. The method of any one of the preceding claims, wherein the plant cell is from a plant in the Rubus family, optionally wherein the plant is Rubus allegheniensis, Rubus occidentalis, Rubus odoratus, or Rubus fruticosus.

35. The method of any one of the preceding claims, wherein the method is genotype-independent.

36. The method of any one of the preceding claims, wherein the method has a transformation efficiency of at least about 4%.

37. A method of producing an edited plant, the method comprising:
introducing a polynucleotide from an Agrobacterium rhizogenes strain into a plant cell of an explant to provide an edited cell, wherein the polynucleotide encodes or comprises at least a portion of an editing system that is capable of modifying a target nucleic acid in the plant cell to provide a modified nucleic acid and thereby an edited cell;
producing a root from the edited cell; and
producing an edited plant from the root.

38. The method of 37, wherein the explant is a shoot node explant (e.g., a shoot stem nodal explant).

39. The method of 37 or 38, wherein the root is a transgenic root and the edited plant is produced from the transgenic root.

40. The method of any one of claims 37-39, wherein the edited plant is a composite plant comprising the transgenic root.

41. The method of 37 or 38, wherein the root is transgene free and comprises cells including the modified nucleic acid, and wherein the edited plant is transgene free and comprises cells including the modified nucleic acid.

42. The method of any one of claims 37-40, further comprising harvesting a mature transgenic root from the edited plant and producing a transgenic plant from the mature transgenic root.

43. The method of any one of claims 37-40, further comprising harvesting a transgene free root that comprises cells including the modified nucleic acid from the edited plant and producing a transgene free edited plant from the transgene free root.

44. A method of transforming a plant cell, the method comprising:
introducing a polynucleotide from a disarmed Agrobacterium rhizogenes strain MSU440 (e.g., Agrobacterium rhizogenes strain DS101.1 and/or Agrobacterium rhizogenes strain comprising a sequence of SEQ ID NO:70) into the plant cell to provide a transformed plant cell, thereby transforming the plant cell.

45. The method of claim 44, further comprising forming a callus (e.g., a transgenic callus) from the transformed plant cell or wherein the plant cell is present in a callus.

46. The method of 44 or 45, wherein the polynucleotide encodes or comprises at least a portion of an editing system that is capable of modifying a target nucleic acid in the plant cell to provide a modified nucleic acid, optionally wherein the polynucleotide encodes or forms a ribonucleoprotein.

47. The method of any one of claims 44-46, wherein the plant cell is a root-forming competent cell.

48. The method of any one of claims 44-47, wherein the plant cell is stably transformed with the polynucleotide.

49. The method of any one of claims 44-48, wherein the polynucleotide is stably expressed in the transformed plant cell and wherein the method further comprises modifying a target nucleic acid in the transformed plant cell to provide an edited, transgenic plant cell including a modified nucleic acid.

50. The method of any one of claims 44-47, wherein the plant cell is transiently transformed with the polynucleotide.

51. The method of any one of claims 44-47 or 50, wherein the polynucleotide is transiently expressed in the transformed plant cell and wherein the method further comprises modifying a target nucleic acid in the transformed plant cell to provide an edited plant cell including a modified nucleic acid.

52. The method of any one of claims 44-51, further comprising producing a root (e.g., a hairy root) from the transformed plant cell and/or callus, optionally wherein the root comprises cells including the modified nucleic acid.

53. The method of claim 52, wherein producing the root from the transformed plant cell and/or callus comprises inducing root formation in the absence of an exogenous plant substance (e.g., plant growth regulators and/or hormones).

54. The method of any one of claims 44-53, further comprising producing a transgenic root from the transformed plant cell, optionally wherein the transgenic root is a transgenic hairy root.

55. The method of claim 54, wherein the transgenic root comprises the polynucleotide, optionally wherein the polynucleotide is transfer DNA (T-DNA) from a plasmid of the disarmed Agrobacterium rhizogenes strain MSU440 (e.g., a tumor-inducing (Ti) plasmid optionally comprising a non-Agrobacterium rhizogenes origin of replication).

56. The method of any one of claims 44-55, further comprising evaluating the efficacy of the polynucleotide, optionally wherein evaluating the efficacy of the polynucleotide comprises evaluating the transformation efficiency and/or genome editing efficiency of the polynucleotide.

57. The method of any one of claims 44-56, wherein the callus (e.g., transgenic callus) and/or root (e.g., transgenic root) are present about 2 weeks to about 4 or 6 weeks after the step of introducing the polynucleotide from the disarmed Agrobacterium rhizogenes strain MSU440.

58. A method for evaluating transgene function in a plant cell, the method comprising:
introducing a polynucleotide from an Agrobacterium rhizogenes strain into the plant cell to provide a transformed plant cell comprising the transgene, optionally wherein the polynucleotide encodes or comprises at least a portion of an editing system that is capable of modifying the target nucleic acid in the plant cell to provide a modified nucleic acid; and
evaluating the transformed plant cell to determine transgene function.

59. The method of claim 58, wherein the evaluating step comprises detecting the presence of hairy roots, detecting the expression of a reporter gene, and/or detecting the presence of a transgenic callus and/or transgenic root.

60. The method of claim 58 or 59, further comprising determining whether the target nucleic acid is modified in the transformed plant cell and/or detecting the presence of the modified nucleic acid in the transformed plant cell.

61. The method of any one of claims 58-60, wherein the evaluating and/or determining step(s) are carried out at about 2 weeks to about 4 or 6 weeks after the step of introducing the polynucleotide from the Agrobacterium rhizogenes strain.

62. A disarmed Agrobacterium rhizogenes strain comprising a deletion in the Ri plasmid, wherein the deletion is in a region of the MSU440 Ri plasmid from base pair 70,000 to base pair 130,000, wherein the base pair number is from the start of the origin of replication of the MSU440 Ri plasmid, or an optimally aligned region thereto for a different Ri plasmid.

63. The disarmed Agrobacterium rhizogenes strain of claim 62, wherein the deletion in the region of the MSU440 Ri plasmid or optimally aligned region thereto is a deletion of about 40,000 base pairs to about 60,000 base pairs, optionally wherein the deletion in the region of the MSU440 Ri plasmid or optimally aligned region thereto is a deletion of about 50,000 base pairs.

64. The disarmed Agrobacterium rhizogenes strain of claim 62 or 63, wherein the deletion in the region of the MSU440 Ri plasmid or optimally aligned region thereto is in a region having a sequence identity of about 70%, 75%, 80%, 85%, 90%, 95%, 96%, 97%, 98%, 99%, or more to SEQ ID NO:67, optionally wherein the region has a sequence of SEQ ID NO:67.

65. The disarmed Agrobacterium rhizogenes strain of any one of claims 62-64, wherein the deletion in the region of the MSU440 Ri plasmid or optimally aligned region thereto has a sequence identity of about 70%, 75%, 80%, 85%, 90%, 95%, 96%, 97%, 98%, 99%, or more to SEQ ID NO:68 (i.e., the Ri plasmid of the disarmed Agrobacterium rhizogenes strain is devoid of a sequence having a sequence identity of about 70%, 75%, 80%, 85%, 90%, 95%, 96%, 97%, 98%, 99%, or more to SEQ ID NO:68), optionally wherein the Ri plasmid of the disarmed Agrobacterium rhizogenes strain is devoid of a sequence of SEQ ID NO:68.

66. The disarmed Agrobacterium rhizogenes strain of any one of claims 62-65, wherein the deletion is in the T-DNA of the Ri plasmid that comprises the rol gene for the MSU440 Ri plasmid or the different Ri plasmid.

67. A disarmed Agrobacterium rhizogenes strain prepared from Agrobacterium rhizogenes strain MSU440 by deleting about 70%, 75%, 80%, 85%, 90%, 95%, 96%, 97%, 98%, 99%, or 100% of SEQ ID NO:67 in the Ri plasmid of the Agrobacterium rhizogenes strain MSU440, wherein the Ri plasmid of the Agrobacterium rhizogenes strain MSU440 has a sequence of SEQ ID NO:69.

68. The disarmed Agrobacterium rhizogenes strain of claim 67, wherein the disarmed Agrobacterium rhizogenes strain is prepared from Agrobacterium rhizogenes strain MSU440 by deleting about 70%, 75%, 80%, 85%, 90%, 95%, 96%, 97%, 98%, 99%, or 100% of SEQ ID NO:68 in the Ri plasmid of the Agrobacterium rhizogenes strain MSU440, optionally wherein the disarmed Agrobacterium rhizogenes strain is prepared from Agrobacterium rhizogenes strain MSU440 by deleting a sequence of SEQ ID NO:68.

69. The disarmed Agrobacterium rhizogenes strain of any one of claims 62-68, wherein the Ri plasmid of the disarmed Agrobacterium rhizogenes strain is devoid of a functional rol gene.

70. The disarmed Agrobacterium rhizogenes strain of any one of claims 62-69, wherein the Ri plasmid of the disarmed Agrobacterium rhizogenes strain is devoid of about 70%, 75%, 80%, 85%, 90%, 95%, 96%, 97%, 98%, 99%, or 100% of one or more of SEQ ID NOs:73-77 and/or devoid of about 70%, 75%, 80%, 85%, 90%, 95%, 96%, 97%, 98%, 99%, or 100% of a sequence encoding one or more of SEQ ID NO:78-82, optionally wherein the Ri plasmid of the disarmed Agrobacterium rhizogenes strain is devoid of a rol gene.

71. The disarmed Agrobacterium rhizogenes strain of any one of claims 62-70, wherein the Ri plasmid of the disarmed Agrobacterium rhizogenes strain is devoid of the T-DNA that contains the rol gene.

72. The disarmed Agrobacterium rhizogenes strain of any one of claims 62-71, wherein the Ri plasmid of the disarmed Agrobacterium rhizogenes strain is devoid of about 70%, 75%, 80%, 85%, 90%, 95%, 96%, 97%, 98%, 99%, or 100% of SEQ ID NO:71 and/or SEQ ID NO:72, optionally wherein the Ri plasmid of the disarmed Agrobacterium rhizogenes strain is devoid of T-DNA.

73. The disarmed Agrobacterium rhizogenes strain of any one of claims 62-72, wherein the Ri plasmid of the disarmed Agrobacterium rhizogenes strain comprises the trans factors of the Ri plasmid of the MSU440 Ri plasmid or the different Ri plasmid, optionally wherein the Ri plasmid of the disarmed Agrobacterium rhizogenes strain comprises the vir genes the MSU440 Ri plasmid or the different Ri plasmid.

74. The disarmed Agrobacterium rhizogenes strain of any one of claims 62-73, wherein the Ri plasmid of the disarmed Agrobacterium rhizogenes strain comprises an additional sequence compared to the MSU440 Ri plasmid or the different Ri plasmid, optionally wherein the additional sequence is about 100, 500, 1,000, 1,500 or 2,000 base pairs to about 2,500, 3,000, or 3,500 base pairs in length.

75. The disarmed Agrobacterium rhizogenes strain of claim 74, wherein the additional sequence is a nucleotide sequence that confers antibiotic resistance (e.g., an antibiotic resistance cassette), optionally wherein the additional sequence is a gentamicin resistance cassette.

76. The disarmed Agrobacterium rhizogenes strain of any one of claims 62-75, wherein the Ri plasmid of the disarmed Agrobacterium rhizogenes strain comprises a sequence having a sequence identity of about 70%, 75%, 80%, 85%, 90%, 95%, 96%, 97%, 98%, 99%, or more to SEQ ID NO:65 and/or a sequence having a sequence identity of about 70%, 75%, 80%, 85%, 90%, 95%, 96%, 97%, 98%, 99%, or more to SEQ ID NO:66, optionally wherein the Ri plasmid of the disarmed Agrobacterium rhizogenes strain comprises a sequence of SEQ ID NO:65 and/or a sequence of SEQ ID NO:66.

77. The disarmed Agrobacterium rhizogenes strain of any one of claims 62-76, wherein the Ri plasmid of the disarmed Agrobacterium rhizogenes strain comprises a sequence having a sequence identity of about 70%, 75%, 80%, 85%, 90%, 95%, 96%, 97%, 98%, 99%, or more to SEQ ID NO:64, optionally wherein the Ri plasmid of the disarmed Agrobacterium rhizogenes strain comprises a sequence of SEQ ID NO:64.

78. The disarmed Agrobacterium rhizogenes strain of any one of claims 62-77, wherein the Ri plasmid of the disarmed Agrobacterium rhizogenes strain comprises a sequence having a sequence identity of about 70%, 75%, 80%, 85%, 90%, 95%, 96%, 97%, 98%, 99%, or more to SEQ ID NO:70, optionally wherein the Ri plasmid of the disarmed Agrobacterium rhizogenes strain comprises a sequence of SEQ ID NO:70.

79. The disarmed Agrobacterium rhizogenes strain of any one of claims 62-78, further comprising a tumor-inducing (Ti) plasmid that comprises a non-Agrobacterium rhizogenes origin of replication, optionally wherein the Ti plasmid comprises an Agrobacterium tumefaciens origin of replication.

80. The disarmed Agrobacterium rhizogenes strain of any one of claims 62-79, wherein the disarmed Agrobacterium rhizogenes strain is Agrobacterium rhizogenes strain DS101.1.

81. The disarmed Agrobacterium rhizogenes strain of any one of claims 62-80, wherein the disarmed Agrobacterium rhizogenes strain is frozen and/or lyophilized.

82. A composition comprising the disarmed Agrobacterium rhizogenes strain of any one of claims 62-81.
